(19) United States
(12) Patent Application Publication
(10) Pub. No.: US 2026/0234195 A1
(43) Pub. Date: Aug. 13, 2026

(54) ACTIVE IMMUNIZATION AGAINST AMYLOID AND AGING-RELATED DISEASES

(71) Applicant: University of Kansas, Lawrence, KS (US)

(72) Inventors: Jackob MOSKOVITZ, Lawrence, KS (US); Adam S. SMITH, Olathe, KS (US); Fei Philip GAO, Lawrence, KS (US)

(21) Appl. No.: 18/706,173

(22) PCT Filed: Nov. 4, 2022
(86) PCT No.: PCT/US2022/079304

§ 371 Date: Apr. 30, 2024

Related U.S. Application Data
(60) Provisional application No. 63/276,382, filed on Nov. 5, 2021.

(51) Int. Cl. C07K 14/00; A61K 39/00; C12P 21/02

(52) U.S. Cl. CPC C07K 14/00; C12P 21/02; A61K 39/00; C07K 2319/21; C07K 2319/24; C07K 2319/50

(57) ABSTRACT

A hybrid polypeptide can be used to prepare a Met-rich protein includes: a histidine tag region; a solubility promoter region adjacent to the histidine tag region; a methionine-rich region, wherein the solubility promoter region is between the histidine tag and the methionine-rich region; and at least one cleavage site between the solubility promoter region and the methionine-rich region, such that cleavage produces a methionine-rich polypeptide. A method of producing a MetO-rich polypeptide includes: cleaving the Met-rich region from the hybrid polypeptide to produce the Met-rich polypeptide; and oxidizing a plurality of methionine residues in the Met-rich polypeptide to form the MetO-rich polypeptide. A method of immunizing a subject against MetO-containing proteins can use the MetO-rich polypeptides to produce anti-MetO antibodies.

### CROSS-REFERENCE TO RELATED APPLICATIONS

This patent application claims priority to U.S. Provisional Application No. 63/276,382 filed Nov. 5, 2021, which provisional is incorporated herein by specific reference in its entirety.

### U.S. GOVERNMENT RIGHTS

This invention was made with government support under GM110761 awarded by the National Institutes of Health. The government has certain rights in the invention.

### SEQUENCE LISTING

This application contains a Sequence Listing which has been submitted in XML format and is hereby incorporated by reference in its entirety. The XML copy, created on Apr. 8, 2026, is named KU_21-042L-03_371_US_ST26 and is 22,037 bytes in size.

### BACKGROUND

### Field

The present disclosure relates to methionine sulfoxide (MetO)-rich polypeptides to produce anti-methionine sulfoxide antibodies for immunization against methionine sulfoxide-containing proteins. The immunization against methionine sulfoxide-containing proteins can inhibit onset or progression of MetO-containing proteins related diseases, —including neurological diseases, —like Alzheimer's disease (AD).

### Description of Related Art

It is known that an organism can be damaged by oxidative stress. The oxidative stress can be present from production of reactive oxygen species that cause oxidative damage in instances where cellular antioxidant function is insufficient (1). Also, oxidative stress can be a cause of neurodegenerative diseases as well as diseases associated with aging (2; 3; 4). One common disease, Alzheimer's disease (AD), results from lipid peroxidation and nucleic and protein oxidation levels that are increased (5). Disease states that are associated with AD may be present with extracellular amyloid plaques, fibrillar amyloid-beta (Aβ), and neurofibrillary tangles (6). Additionally, oxidative stress can lead to unfavorable immunological responses, such as inflammation, activated microglia, and astrocytes (6). It is known that amyloid plaques can lead to AD (7). It is thought that neurotoxic soluble Aβ oligomers can result in the formation of plaques and tangles (8; 9). The mitochondrion is sensitive to oxidative stress and deficiencies can result in oxidative damage to the neuronal cell (10). Previously, some antioxidant-based therapies have been suggested to treat AD (10; 11).

It is known that Aβ contains a single methionine at position 35 of the protein sequence, which is in a hydrophobic region, and thereby polarity increases upon methionine oxidation (12). The total Aβ plaques can have up to 50% methionine sulfoxide (MetO)-Aβ during oxidative stress (13; 14; 15). It is thought that the immune system and upregulation of methionine sulfoxide reductase type A (MsrA) can provide cellular antioxidant responses (12; 16; 17; 18).

It is also known that older humans are more susceptible to oxidative stress, in part due to a decrease in activity of MsrA and other antioxidants (19). Accordingly, it is thought that Aβ and possibly other MetO-containing proteins increase toxicity towards neurons. The higher solubility of Aβ can contribute to the toxicity due to an increase in mobility and access to neurons in the brain (12). Accordingly, it may be advantageous to improve clearance of Aβ from brain and clearance of other extracellular MetO-containing proteins in order to inhibit AD and cognitive decline.

Previously, Aβ-derived antigens have been used for immunization, and anti-Aβ antibodies for have been used for passive immunization, which may reduce Aβ burden (Aduhelm™, Biogen). It is known that injecting a rabbit with MetO-rich protein (the “Antigen”) results in the production of an antibody that is able to bind and recognize a MetO moiety in a protein (U.S. Pat. No. 8,409,824, which is incorporated herein by specific reference). This antigen was initially produced as a recombinant 6His-tag protein that was purified and oxidized with H₂O₂ as previously described in the patent above. However, there is still room for improvement using immunization for inhibiting problems with Aβ or other MetO-containing proteins. Therefore, it would be advantageous to have an improved method of obtaining a high yield purified MetO-rich antigen that can be used in an immunization treatment for inhibiting problems associated with Aβ or other MetO-containing proteins.

### SUMMARY

In some embodiments, a plasmid can have a nucleic acid sequence that encodes for a hybrid polypeptide. The hybrid polypeptide can include: a histidine tag region; a solubility promoter region adjacent to the histidine tag region; a methionine-rich region, wherein the solubility promoter region is between the histidine tag and the methionine-rich region; and at least one cleavage site between the solubility promoter region and the methionine-rich region, such that cleavage produces a methionine-rich polypeptide. In some aspects, the plasmid can include the encoded hybrid polypeptide that has a polypeptide sequence of SEQ ID NO: 1 or SEQ ID NO: 12, or at least 80%, 90%, 95%, or 99% identity therewith. In some aspects, the plasmid can be characterized by at least one of: the histidine tag is N-terminal and has a plurality of consecutive histidine residues and the solubility promoter region is MBP in a 6His-MBP polypeptide having a SEQ ID NO: 11, or at least 80%, 90%, 95%, or 99% identity therewith; or the methionine-rich region is SEQ ID NO: 2, SEQ ID NO: 3, SEQ ID NO: 8 or SEQ ID NO: 13, or at least 80%, 90%, 95%, or 99% identity therewith.

In some embodiments, a hybrid polypeptide can include: a histidine tag region; a solubility promoter region adjacent to the histidine tag region; a methionine-rich region, wherein the solubility promoter region is between the histidine tag and the methionine-rich region; and at least one cleavage site between the solubility promoter region and the methionine-rich region, such that cleavage produces a methionine-rich polypeptide.

In some embodiments, an anti-methionine sulfoxide antibody is provided that binds with a methionine sulfoxide-rich polypeptide, wherein the antibody binds with a polypeptide region having MetO residues, such as for example, with a sequence of SEQ ID NO: 2, 3 or 8, or at least 80%, 90%, 95%, or 99% identity therewith.

In some embodiments, an immunization composition can include a methionine sulfoxide-rich polypeptide antigen derived from a methionine sulfoxide-rich polypeptide, which can for example include the sequence of SEQ ID NO: 2, 3, 8, or 13, or at least 80%, 90%, 95%, or 99% identity therewith, wherein the methionine sulfide-rich polypeptide includes a majority of methionine residues being oxidated. The immunizing composition can also include an adjuvant and other pharmaceutically acceptable excipients and carriers.

In some embodiments, a method of producing a methionine-rich polypeptide can include: providing the plasmid of one of the embodiments that encodes for a hybrid polypeptide; producing the hybrid polypeptide from the plasmid; and cleaving a methionine-rich region from the hybrid polypeptide to produce the methionine-rich polypeptide.

In some embodiments, a method of producing a methionine sulfoxide-rich polypeptide can include: providing the plasmid of one of the embodiments that encodes for a hybrid polypeptide; producing the hybrid polypeptide from the plasmid; cleaving the methionine-rich region from the hybrid polypeptide to produce the methionine-rich polypeptide; and oxidizing a plurality of methionine residues in the methionine-rich polypeptide to form the methionine sulfoxide-rich polypeptide.

In some embodiments, a method of immunizing a subject against methionine sulfoxide-containing proteins can include: obtaining the methionine sulfoxide-rich polypeptide; and immunizing the subject against the methionine sulfoxide-containing proteins by administration of the methionine sulfoxide-rich polypeptide. In some aspects, the immunizing of the subject is sufficient to at least one of: inhibit onset or progression of a neurodegenerative disease, which is optionally Alzheimer's disease; inhibit onset or progression of a learning disorder; inhibit onset or progression of memory loss; produce anti-methionine sulfoxide antibodies; improving cognitive function; or increasing clearance of methionine sulfoxide-containing proteins.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1A shows a gel that indicates the MetO-rich protein (MetO antigen) was produced and purified according to the procedures described herein.

FIG. 1B shows a dot-blot analysis using the MetO-rich protein as the loaded protein (MetO antigen) and rabbit anti-MetO antibody (1:1000 dilution) as the primary antibody.

FIG. 1C shows a dot-blot analysis using the MetO-rich protein, as the loaded protein (MetO antigen), probed with plasma moiety (used as the primary antibody, 1:100 dilution, 1 h incubation time) from 10-month-old mice.

FIG. 2A shows a graph that indicates the MetO antigen-immunized APP/PS1 mice have a novel arm preference in a forced Y-maze test but control APP/PS1 do not, displaying improved short-term memory after vaccination.

FIG. 2B shows a graph that indicates that arm crossing is not affected by vaccination, indicating age-typical locomotion.

FIG. 2C shows a representation of the Morris Water Maze (MWM) analyses.

FIG. 2D shows a graph with a statistical presentation of the significant effect on the antigen-immunized mice's reduced escape latency during the second day of training, while the control mice do not improve in this task until day 3.

FIG. 2E shows a graph that indicates that MetO Antigen-immunized mice also spend more time swimming in the target quadrant (after the platform is removed from the pool) compared to controls, who are only performing by chance.

FIG. 3A shows images of the hemisphere brains of post-mortem mice (control and anti-gen-immunized) that were dissected, and immunohistochemistry analyses were performed to detect Aβ₄₂ in the three selected brain regions (HPC, hippocampus; RSC, retrosplenial cortex, and EC, and entorhinal cortex).

FIG. 3B shows a graph of the quantification of the data presented in FIG. 3A using the NIH-Image J program.

FIG. 3C shows a graph of quantification of Aβ₄₂ in blood-plasma.

FIG. 3D shows a graph that indicates Aβ₄₂ in post-mortem brain extracts of whole brain.

FIG. 3E shows images of the thioflavin-S staining of the post-mortem mouse brains in the three selected brain regions.

FIG. 3F shows a graph of the quantification of the data depicted in FIG. 3E using the NIH Image-J program.

FIG. 4A shows images of hemisphere brains of post-mortem mice (control and antigen-immunized) that were dissected, and immunohistochemistry analyses were performed to detect MetO proteins in the three selected brain regions.

FIG. 4B shows a graph of the quantification of the data presented in FIG. 4A using the NIH-Image J program.

FIG. 5A shows images of the hemisphere brains of post-mortem mice (control and antigen-immunized) that were dissected, and immunohistochemistry analyses were performed to detect Iba1 in the three selected brain regions.

FIG. 5B shows a graph of the quantification of the data presented in FIG. 5A using the NIH-Image J program.

FIG. 6A shows images of hemisphere brains of post-mortem mice (control and antigen-immunized) that were dissected, and immunohistochemistry analyses were performed to detect Nrf2.

FIG. 6B shows quantification of the data presented in FIG. 6A by manually counting the nuclear Nrf2 signal per same-size area in the three selected brain regions.

FIG. 7A shows a Western blot analysis of an immunoprecipitation (IP), using rabbit anti-MetO antibody, of extracellular proteins of human brains samples of late stage of AD and Non-AD control brains, probed with primary mouse anti-human apolipoprotein J (Apo-J) antibody.

FIG. 7B shows another gel of a Western blot analysis that was performed with extracellular brain protein extracts obtained from the brains in FIG. 7A (using the same mouse anti-human Apo-J antibody), which showed no significant difference in the expression of Apo-J between the AD and the Non-AD brains.

FIG. 8A shows a Western blot analysis of an IP, using rabbit anti-MetO antibody, of 5FAD mouse (an AD mouse model) brain samples of late stage of AD and Non-AD control mouse brains, probed with primary goat anti-mouse apolipoprotein Apo-J antibody.

FIG. 8B shows a Western blot analysis of brain protein extracts was obtained for the brains described in FIG. 8A (using the same goat anti-mouse Apo-J antibody, which showed no significant difference in the expression of Apo-J between the AD and the control mouse brains. Levels of β-actin serve as gel-loading controls.

FIG. 9 shows the sequence of the MalE gene in the pMAL-c6T vector (e.g., MBP region).

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology is related to an improved nucleic acid system and protocol for generating a methionine (Met) polypeptide that can be processed into a methionine sulfoxide (MetO) antigen that contains a plurality of MetO residues. The MetO antigen can be used for immunization against MetO-containing proteins to reduce those MetO-containing proteins, such as aberrant beta amyloid (amyloid beta; Aβ) or others, in a subject. Accordingly, the MetO antigen can be used in immunization to reduce Aβ burden (e.g., has a MetO residue) in a subject, such as a patient with AD. The MetO antigen can cause the generation of anti-MetO antibodies, which can be used for clearance of MetO-containing proteins, such as Aβ and other extracellular MetO-containing proteins (MetO proteins). Accordingly, the present technology provides for active immunization, using a MetO antigen. In some aspects, the MetO antigen can be derived from an oxidized Zea mays Met-rich protein, which MetO antigen can be used for anti-MetO immunization. Additionally, the anti-MetO antibody can target other extracellular MetO proteins to be cleared.

In some embodiments, immunization with the MetO antigen can be used to prevent or treat neurological disorders that arise from MetO proteins. Accordingly, immunization with the MetO antigen can provide protection against AD, by targeting MetO-Aβ. Additionally, immunization with the MetO antigen can be used generally for reducing memory decline and reducing learning difficulties as well as boosting cognitive performance. As a result, the immunization with the MetO antigen can be used to improve memory and improve learning, which is evidenced with the data provided herein.

A MetO-rich protein (the “MetO antigen”) is taught in U.S. Pat. No. 8,409,824, which is produced as a recombinant 6His-tag protein that is purified and oxidized with H₂O₂. However, it has been found to be difficult to obtain sufficient Met-rich polypeptide for conversion into a MetO antigen for use in therapeutics. Due to limitation in the production yield of the Met-rich polypeptide, the present technology provides a method that uses a Maltose-Binding Protein (MBP) polypeptide (e.g., or other solubility promoter entity) that contains the 6His-tag moiety. That is, the MBP polypeptide is in the same region as the 6His-MBP tag. This change to the 6His-MBP tag caused a surprising and unexpected dramatic increase in the yield of the soluble recombinant 6His-MBP-Met-rich polypeptide (e.g., up to 10 mg of protein/Liter). Following affinity purification through the 6His binding, the pure MBP-Met-rich polypeptide was digested with trypsin that degraded the MBP moiety from the desired Met-rich polypeptide. The degradation cleaves the MBP polypeptide from the Met-rich polypeptide by cleaving the structure at lysine and arginine residues to form small peptides. Trypsin cleaves the peptide bond between the carboxyl group of arginine or the carboxyl group of lysine and the amino group of the adjacent amino acid. The rate of cleavage occurs more slowly when the lysine and arginine residues are adjacent to acidic amino acids in the sequence or cystine. Cleavage does not occur when lysine or arginine is followed by proline. The sites of trypsin cleavage can be limited to arginine peptide bonds by succinylation or citraconylation prior to trypsin digestion. This allows for the trypsin to now cleave the MBP polypeptide from the Met-rich polypeptide due to the selection of the amino acids to avoid arginine or lysine in the Met-rich polypeptide. Thus, the Met-rich polypeptide is available for oxidation and subsequent use as an antigen.

In some embodiments, the Met-rich region is configured to have a lack of lysine and arginine residues in the Met-rich moiety. In some instances, an exception to no lysine is when there is one N-terminal lysine residue that is only partly cleaved by the trypsin. This partially cleaved N-terminal lysine is allowable as the resulting Met-rich polypeptide is not cleaved or compromised in any way, and thereby does not affect the integrity of the Met-rich region of the polypeptide. Only 1, 2, 3, 4 or 5 residues in the Met-rich polypeptide can be arginine or lysine, preferably one; however, a small number may be usable with some amino acid sequences. For example, any lysine or arginine in the Met-rich region may be adjacent acidic amino acids, cystine, or proline in order to inhibit cleaving at that lysing or arginine. This procedure allows the retention of the functionality of the Met-rich region, which is substantiated herein.

Due to the use of the MBP polypeptide and cleavage with trypsin, the resulting structure is now different and new, such as described herein.

After cleaving, the Met-rich polypeptide is obtained with a remaining intact pre-antigen section that has a higher percentage of Met. The Met-rich polypeptide can be further purified to homogeneity using a size exclusion SEC75 column and then oxidized with H₂O₂, resulting in the desired pure MetO-rich polypeptide (e.g., details of the polypeptide sequence and its purification steps are described herein). The MetO-rich polypeptide may be considered to be a MetO-rich recombinant protein, such as when based on a naturally occurring Met-rich protein.

It was found that the fusion of the 6His-MBP polypeptide with the Met-rich polypeptide provided a high yield of the hybrid polypeptide, which was obtained in high quantity. Using trypsin for degradation provided a new and novel Met-rich polypeptide that can be used to form the MetO antigen by oxidation (e.g., hydrogen peroxide). The resulting MetO antigen has surprising and unexpected superior results in functioning as an antigen for MetO immunological function.

In some embodiments, the MetO antigen can be used to reduce amyloid plaque in a subject. The immunization of AD-model mice with a MetO antigen is shown to reduce the amyloid-plaque burden by about 30% (e.g., 28%). These data suggest that MetO-proteins play a role in modulation of AD markers in both mice and human. However, the extent and the specific effect of MetO-proteins on brain function is not known. Accordingly, it is hypothesized that lowering the level of MetO-proteins in blood and brain through immunization with the MetO antigen can be used to treat, prevent, inhibit, or otherwise alleviate various disease states that are related to MetO-proteins, such as Aβ. Accordingly, the reduction of amyloid plaque may be used to treat neurodegenerative diseases, such as AD. Also, reduction in amyloid plaque may reduce memory loss and learning deficits. In some aspects, the MetO antigen can be used to treat any subject in order to treat, prevent, inhibit or otherwise alleviate neurodegenerative disease (AD), memory loss, and learning deficit. In some aspects, the MetO antigen can be used to treat, prevent, inhibit or otherwise alleviate memory loss and learning deficit, whether or not associated with AD.

In some embodiments, the amyloid plaque burden can be reduced after immunization with the MetO antigen. The reduction can be at least a 10% reduction, at least 20% reduction, at least 25% reduction, at least 30% reduction, at least 35% reduction, or even a larger reduction, such as 50% or more with optimization.

In some embodiments, the amount of MetO protein in blood or brain can be reduced after immunization with the MetO antigen. The reduction of MetO protein can be at least 5%, at least 10% reduction, at least 20% reduction, at least 25% reduction, at least 30% reduction, at least 35% reduction, or even a larger reduction, such as 50% or more with optimization.

In some embodiments, the MetO antigen can be used in a treatment as a prophylactic for the brain to be protective against neurodegenerative diseases, like AD. As such, prior to onset of neurodegenerative disease, the MetO antigen can be used as a prophylactic for reduction of memory or learning deficits, which are linked to MetO proteins. In some aspects, the MetO antigen can be used in order to treat, prevent, inhibit or otherwise alleviate memory loss and learning deficit associated with MetO proteins, whether or not associated with a neurodegenerative disease. In some embodiments, the MetO antigen can be used in an immunization to inhibit the toxic effect of oxidative stress in a tissue, like the brain, which is mediated by reactive oxygen species. The MetO antigen can be used to immunize the subject against accumulation of Met35-oxidized Aβ. In some aspects, the MetO antigen can inhibit the accumulation of methionine-oxidized residues of other extracellular proteins (e.g. Apo-J). Accordingly, the MetO antigen can be used in an immunization to inhibit or stop the build-up of proteins having MetO (e.g. Apo-J and Aβ). Thus, prophylactic protection can be provided before onset of a neurodegenerative disease.

In some embodiments, the MetO antigen provides a positive effect on brain function by the described immunization. Moreover, the acquired data suggest a novel use of the described immunization as a treatment or prophylactic against memory loss and learning capabilities that are affected by MetO protein accumulation in oxidative-stress related diseases, like AD. The MetO antigen produces an anti-MetO antibody. Therefore, the MetO antibody can be used in methods for treating and/or inhibiting or otherwise ameliorating memory loss and/or learning difficulties. That is, this MetO antibody can be used as a therapy for a subject having onset of memory loss or learning difficulties, or a prophylactic to a subject wanting to inhibit or prevent such onset of memory loss or learning difficulties. Thus, the MetO antigen can be used to generate the ani-MetO antibody in order to provide the therapeutic benefits described herein.

In some embodiments, immunization with the MetO antigen can improve cognitive function by clearance of potentially MetO-containing proteins (including Aβ) from brain and blood by the immune system. That is, the subject's own immune system can become more reactive against MetO-containing proteins (e.g., with anti-MetO antibody), and thereby more MetO-containing proteins are removed through the function of the immune system. As a result, the cognitive function can be improved due to the reduction of MetO-containing proteins, whether or not associated with a neurodegenerative disease.

In some embodiments, the present technology discloses methods of making antibodies that bind to MetO-containing proteins. The antibodies are made in response to the MetO antigen, prepared as described herein, being administered to a subject that has an antibody generating immunological response. The present technology discloses methods of making the MetO antigen, which can be a MetO-rich recombinant protein, and providing the MetO antigen to a subject that thereby causes production of the anti-MetO antibody. The present invention provides methods of using the MetO antigen for immunization of a subject, which immunization produces the anti-MetO antibodies that bind to MetO-containing proteins, which induces clearance. However, it is possible that the anti-MetO antibodies can be generated, purified, and administered as a therapeutic to treat or prevent MetO protein-associated disease states.

In some embodiments, the present technology provides a non-Aβ protein-based active immunization by using the MetO-rich protein (MetO antigen is not based on Aβ protein) that can provide protection against cognitive decline. As a result, immunogenicity against the precursor proteins that form into amyloids is likely avoided due to the MetO antigen being based on a different type of protein (e.g., not a human protein). The utility of such active immunization with the MetO-rich protein is better in comparison to other types of immunizations designed to treat AD in humans. In some aspects, immunization with the MetO-rich protein can provide a reduction in Aβ deposits and plaque burden in brain, especially in the hippocampal and cortical regions. Accordingly, immunization can be used for preventing the development/progression of any disease state related to Aβ deposits. Accordingly, immunization with MetO antigen can provide a reduction in the levels of the most neurotoxic form of Aβ (i.e., Aβ₄₂ types) in blood-plasma, whole brain, and specific brain regions. The MetO antigen can be used to provide lower peripheral blood-plasma levels of Aβ₄₂.

In some embodiments, a subject may exhibit detectable or increased levels of MetO-proteins in astrocytes in hippocampus (HPC), retrosplenial cortex (RSC), and entorhinal cortex (EC) brain regions, which can be significantly diminished upon MetO antigen immunization. This reduction of MetO proteins may be attributed to the ability of the MetO antigen to provide anti-MetO antibodies that target MetO-rich proteins for immunization, which can reduce oxidative stress that in-turn lowers the cellular level of protein-MetO.

In some embodiments, treatment with the MetO antigen can cause Nrf2 to be predominantly located in the nucleus of neuronal cells of brains. Accordingly, the MetO antigen can be used to provide a brain of a treated subject with a stronger antioxidant defense. For example, the MetO antigen can provide protection against methionine oxidation in astrocytes. In some aspects, the MetO antigen can also cause increased nuclear localization of the antioxidant regulator, Nrf2. The antigen vaccination can promote cellular antioxidant protection in the brain, in part, through the activation of Nrf2.

In some embodiments, active immunization with MetO-rich antigen can be used to inhibit short-term and long-term memory loss. The use of MetO antigen-immunization can provide a reduction in total Aβ₄₂ levels, both in blood-plasma and whole brain, which can inhibit memory loss in a subject. Accordingly, the decline in Aβ₄₂ levels can also be accompanied by a reduction of amyloid plaque burden in the hippocampal and cortical regions of the brain in order to provide the effect. Thus, short-term and long-term memory loss may be inhibited, and overall memory may be improved.

In some embodiments, the MetO antigen can be used for reducing activated microglia along with the reduction of MetO-protein levels in astrocytes. The MetO antigen can be used to reduce the levels of activated microglia in the brain. Increased level of activated microglia is a marker for neurodegeneration processes, prompted by enhanced levels of beta amyloid in AD. As such, reduction can be used to assess treatment of AD.

In some embodiments, the MetO antigen can be used to generate anti-MetO antibodies that target oxidized Apo-J that is oxidized in its methionine moiety. It has now been discovered that Apo-J (e.g., Clusterin) protein is oxidized in its methionine moiety in human Alzheimer's disease. The MetO-containing Apo-J can been shown to be present in post mortem brains and in an Alzheimer's disease model mice compared with controls. Apo-J is a secreted protein that acts as a chaperone and binds beta amyloid. Binding by Apo-J is supposed to prevent beta amyloid aggregation. Thus, it is hypothesized that the oxidation of Apo-J methionine(s) may compromise its function, and thereby not able to bind beta amyloid. Accordingly, the anti-MetO antibody may remove the oxidized Apo-J, and thereby foster the denovo production and secretion of native non-oxidized Apo-J. Accordingly, the MetO antigen may indirectly lead to production of non-oxidized Apo-J, which can then provide for the non-oxidized Apo-J to bind with beta amyloid. In turn, the new Apo-J can better function in preventing the aggregation of beta amyloid, thereby preventing the progression of Alzheimer's disease.

According to the methods of the invention, the peptide may be administered to subjects by a variety of administration modes, including by intradermal, intramuscular, subcutaneous, intravenous, intra-atrial, intra-articular, intraperitoneal, parenteral, oral, rectal, intranasal, intrapulmonary, and transdermal delivery, or topically to the eyes, ears, skin or mucous membranes.

In certain embodiments of the invention, the MetO polypeptides or pharmaceutical composition with or without a co-stimulatory molecule (adjuvant) are delivered to a common or adjacent target site in the subject (e.g., blood, brain), for example to a specific target tissue (brain) or cell population in which the vaccine formulation is intended to elicit an immune response. Typically, when the peptide or pharmaceutical composition and the optional co-stimulatory molecule are administered separately, they are delivered to the same or closely proximate site(s), for example to a single target tissue or to adjacent sites that are structurally or fluidly connected with one another (e.g., to allow direct exposure of the same cells, e.g., fluid flow transfer, dissipation or diffusion through a fluid or extracellular matrix of both vaccine agents). Thus, a shared target site for delivery of antigen and co-stimulatory molecule can be a common surface (e.g., a mucosal, basal or luminal surface) of a particular target tissue or cell population, or an extracellular space, lumen, cavity, or structure that borders, surrounds or infiltrates the target tissue or cell population, such as the brain.

For prophylactic and treatment purposes, the MetO antigen with or without a co-stimulatory molecule may be administered to the subject separately or together, in a single bolus delivery, via continuous delivery (e.g., continuous intravenous or transdermal delivery) over an extended time period, or in a repeated administration protocol (e.g., on an hourly, daily or weekly basis). The various dosages and delivery protocols contemplated for administration of peptide and co-stimulatory molecule, in simultaneous or sequential combination, are immunogenically effective to inhibit the occurrence or alleviate one or more symptoms of the target disease (e.g., cancer) in the subject. An “immunogenically effective amount” of the MetO antigen thus refers to an amount that is, in combination, effective, at dosages and for periods of time necessary, to elicit a specific T lymphocyte mediated immune response, e.g., a B/T lymphocyte-mediated immune response. B lymphocytes are needed for antibody production and T lymphocytes work as T-helper cells in this process. This response can be determined by conventional assays for B/T-cell activation, including but not limited to assays to detect proliferation, specific cytokine activation and/or cytolytic activity.

For prophylactic and therapeutic use, MetO antigens can be formulated with a “pharmaceutical acceptable carrier”. As used herein, “pharmaceutically acceptable carrier” includes any and all solvents, dispersion media, coatings, antibacterial and antifungal agents, isotonic and absorption enhancing or delaying agents, and other excipients or additives that are physiologically compatible. In specific embodiments, the carrier is suitable for intranasal, intravenous, intramuscular, intradermal, subcutaneous, parenteral, oral, transmucosal or transdermal administration. Depending on the route of administration, the active compound may be coated in a material to protect the compound from the action of acids and other natural conditions which may inactivate the compound.

A MetO polypeptide vaccine may be administered to the subject in the form of a peptide solution per se or a combination of a peptide with an appropriate auxiliary agent using an injector. Alternatively, the MetO polypeptide vaccine may be percutaneously administered through mucous membrane by, for instance, spraying the solution. The unit dose of the MetO polypeptide typically ranges from about 0.01 mg to 100 mg, more typically between about 100 micrograms to about 5 mg, which may be administered, one time or repeatedly, to a patient.

In preparing pharmaceutical compositions of the present invention, it may be desirable to modify the MetO polypeptide antigen, or to combine or conjugate the peptide with other agents, to alter pharmacokinetics and biodistribution. A number of methods for altering pharmacokinetics and biodistribution are known to persons of ordinary skill in the art. Examples of such methods include protection of the proteins, protein complexes and polynucleotides in vesicles composed of other proteins, lipids (for example, liposomes), carbohydrates, or synthetic polymers. For example, the vaccine agents of the invention can be incorporated into liposomes in order to enhance pharmacokinetics and biodistribution characteristics.

In some embodiments, the MetO antigen can be formulated in a pharmaceutical composition with an adjuvant for immunization purposes. The adjuvant can be of any type that facilitates an immune response to the antigen. There are many known adjuvants in widespread use, including aluminium salts, small molecules, oils and virosomes. Example adjuvants include potassium alum, aluminium hydroxide, aluminium phosphate, calcium phosphate hydroxide, and others.

### Embodiments

Embodiment 1—A plasmid can have a sequence that encodes for a hybrid polypeptide, the hybrid polypeptide comprising: a histidine tag region; a solubility promoter region adjacent to the histidine tag region; a methionine-rich region, wherein the solubility promoter region is between the histidine tag and the methionine-rich region; and at least one cleavage site between the solubility promoter region and the methionine-rich region, such that cleavage produces a methionine-rich polypeptide.

The plasmid of one of the embodiments, wherein at least one of: the histidine tag has a plurality of (e.g., at least 3, 4, 5, 6, etc.) consecutive histidine residues; the solubility promoter region is selected from MBP, GST, SUMO, or GB1; the methionine-rich region has at least 10%, 15%, 20%, 25%, 30%, 35%, 40%, 45%, or 50% amino acids being methionine; or the at least one cleavage site is a protease cleavage site.

The plasmid of one of the embodiments can have at least one of: the histidine tag is N-terminal and has at least 4 or less than 20 consecutive histidine residues; the solubility promoter region is MBP; the methionine-rich region has at least 24% amino acids being methionine; or the at least one cleavage site is a trypsin cleavage site. In some aspects, the methionine-rich region can include at least 10% amino acids being methionine residues, or at least 15%, or at least 20%, or at least 25%, or at least 30%, or at least 35%, or at least 40%, or at least 50% of the amino acids being methionine residues.

The plasmid of one of the embodiments can include the encoded hybrid polypeptide having a polypeptide sequence of SEQ ID NO: 1 or SEQ ID NO: 12, or at least 80%, 90%, 95%, or 99% identity therewith.

The plasmid of one of the embodiments can include at least one of: the histidine tag is N-terminal and has 6 consecutive histidine residues and the solubility promoter region is MBP in a His-MBP polypeptide having a SEQ ID NO: 11, or at least 80%, 90%, 95%, or 99% identity therewith; or the methionine-rich region is SEQ ID NO: 2, 3, 8, or 13, or at least 80%, 90%, 95%, or 99% identity therewith.

A cell can include the plasmid of one of one of the embodiments, which can be in a cell culture or an organism, or an organ or vascular network or tissue or biological fluid of an organism.

A hybrid polypeptide can include: a histidine tag region; a solubility promoter region adjacent to the histidine tag region; a methionine-rich region, wherein the solubility promoter region is between the histidine tag and the methionine-rich region; and at least one cleavage site between the solubility promoter region and the methionine-rich region, such that cleavage produces a methionine-rich polypeptide.

The hybrid polypeptide of one of the embodiments can include at least one of; the histidine tag has at least 4 consecutive histidine residues; the solubility promoter region is selected from MBP, GST, SUMO, or GB1; the methionine-rich region has at least 20% amino acids being methionine; or the at least one cleavage site is a protease cleavage site.

A hybrid polypeptide of one of the embodiments can include at least one of: the histidine tag is N-terminal and has about 4-10 (e.g., 6) consecutive histidine residues; the solubility promoter region is MBP; the methionine-rich region has at least 24% amino acids being methionine; or the at least one cleavage site is a protease cleavage site.

A hybrid polypeptide of one of the embodiments can include a polypeptide sequence of SEQ ID NO: 1 or SEQ ID NO: 12, at least 80%, 90%, 95%, or 99% identity therewith.

A hybrid polypeptide of one of the embodiments can include at least one of: the histidine tag is N-terminal and has about 6 consecutive histidine residues and the solubility promoter region is MBP in a His-MBP polypeptide having a SEQ ID NO: 11, or at least 80%, 90%, 95%, or 99% identity therewith; or the methionine-rich region is SEQ ID NO: 2, 3, 8, or 13, or at least 80%, 90%, 95%, or 99% identity therewith.

A methionine sulfoxide-rich polypeptide can be prepared by cleaving a methionine-rich polypeptide from a hybrid polypeptide having a histidine-solubility promoter region, and oxidizing the methionine-rich polypeptide. The percentage of methionine residues that are oxidized to methionine sulfoxide can be at least 50%, at least 60%, at least 75%, at least 80%, at least 90%, or at least 95% methionine sulfoxide residues of prior methionine residues.

A method of preparing an antigen can include: obtaining a MBP-containing Met-rich polypeptide; digesting the MBP-containing Met-rich polypeptide with trypsin; and obtaining a Met-rich polypeptide without the MBP region.

A method of preparing an antigen can include: providing a plasmid that encodes for a hybrid polypeptide having a His-MBP region and a Met region; digesting the His-MBP region from the Met region with trypsin to obtain a Met-rich polypeptide; and treating the Met-rich polypeptide to obtain a MetO-rich polypeptide.

The method of one of the embodiments can include obtaining up to 10 mg of the His-MBP-Met polypeptide per liter, or up to 1 mg, 2 mg, 4 mg, 5, mg, 6, mg, 8, mg, or 10 mg per liter, or any range therebetween.

The method of one of the embodiments can include purifying the His-MBP-Met polypeptide with affinity chromatography. The method can include using any histidine tag purification technique that is available.

The method of one of the embodiments can include using a sufficient amount of trypsin to degrade a MBP polypeptide from a Met-rich polypeptide.

A method of producing a methionine-rich polypeptide can include: providing the plasmid of one of the embodiments; producing the hybrid polypeptide from the plasmid; and cleaving the methionine-rich region from the hybrid polypeptide to produce the methionine-rich polypeptide.

A method of producing a methionine-rich polypeptide can include: providing the hybrid polypeptide of one of the embodiments; and cleaving the methionine-rich region from the hybrid polypeptide to produce the methionine-rich polypeptide.

A method of producing a methionine sulfoxide-rich polypeptide can include: providing the plasmid of one of the embodiments; producing the hybrid polypeptide from the plasmid; cleaving the methionine-rich region from the hybrid polypeptide to produce the methionine-rich polypeptide; and oxidizing a plurality of methionine residues in the methionine-rich polypeptide to form the methionine sulfoxide-rich polypeptide.

A method of producing a methionine-rich polypeptide can include: providing the hybrid polypeptide of one of the embodiments; cleaving the methionine-rich region from the hybrid polypeptide to produce the methionine-rich polypeptide; and oxidizing a plurality of methionine residues in the methionine-rich polypeptide to form the methionine sulfoxide-rich polypeptide.

The method of one of the embodiments can include at least one of: performing size exclusion chromatography with the Met-rich polypeptide or MetO-rich polypeptide; or treating the Met-rich polypeptide with hydrogen peroxide to obtain the MetO-rich polypeptide.

An anti-methionine sulfoxide antibody can be obtained that binds with a methionine sulfoxide-rich polypeptide that includes the sequence of SEQ ID NO: 2, 3, 8, or 13, or at least 80%, 90%, 95%, or 99% identity therewith.

An immunization composition comprising: a methionine sulfoxide-rich polypeptide antigen derived from a methionine sulfoxide-rich polypeptide that includes the sequence of SEQ ID NO: 2, 3, 8, or 13, or at least 80%, 90%, 95%, or 99% identity therewith, wherein the methionine sulfide-rich polypeptide includes a majority of methionine residues being oxidized; and an adjuvant. The oxidation percentage can be the percentage of Met residues oxidized to MetO residues, which can be at least 10%, at least 20%, at least 30%, at least 40%, at least 50%, at least 60%, at least 70%, at least 80%, at least 90%, or about 99% or more. Preferably, most of the Met residues are oxidized to MetO residues.

A method of immunizing a subject against methionine sulfoxide-containing proteins can include: obtaining the methionine sulfoxide-rich polypeptide of an embodiment; and immunizing the subject against the methionine sulfoxide-containing proteins by administration of the methionine sulfoxide-rich polypeptide. The immunization can generate an anti-MetO antibody that targets MetO-containing proteins for clearance, which can provide for this effect.

A method of treating or inhibiting Alzheimer's Disease can include: obtaining the methionine sulfoxide-rich polypeptide of an embodiment; and administration of the methionine sulfoxide-rich polypeptide to a subject sufficient for immunizing the subject against the methionine sulfoxide-containing proteins. The immunization can generate an anti-MetO antibody that targets MetO-containing proteins for clearance, which can provide for the treatment or prevention of AD.

A method of treating or inhibiting a neurodegenerative disease can include: obtaining the methionine sulfoxide-rich polypeptide of an embodiment; and administration of the methionine sulfoxide-rich polypeptide to a subject sufficient for immunizing the subject against the methionine sulfoxide-containing proteins. For example, the therapeutic benefit can be from the anti-MetO antibody targeting MeO proteins for clearance.

A method of treating or inhibiting a learning disorder can include: obtaining the methionine sulfoxide-rich polypeptide of an embodiment; and administration of the methionine sulfoxide-rich polypeptide to a subject sufficient for immunizing the subject against the methionine sulfoxide-containing proteins. Here, the therapeutic benefit can be from the anti-MetO antibody targeting MeO proteins for clearance.

A method of treating or inhibiting memory loss can include: obtaining the methionine sulfoxide-rich polypeptide of an embodiment; and administration of the methionine sulfoxide-rich polypeptide to a subject sufficient for immunizing the subject against the methionine sulfoxide-containing proteins. The therapeutic benefit can be from the anti-MetO antibody targeting MeO proteins for clearance.

A method of producing anti-methionine sulfoxide antibodies can include: obtaining the methionine sulfoxide-rich polypeptide of an embodiment; and administration of the methionine sulfoxide-rich polypeptide to a subject sufficient for causing an immunization response from the subject against the methionine sulfoxide-containing proteins so as to produce the anti-methionine sulfoxide antibodies. These anti-MetO antibodies can be used to obtain the therapeutic benefits provided herein, whether an active treatment of a disease state due to MetO proteins, or a prophylactic that inhibits a disease state by increasing clearance of MetO proteins.

A method of improving cognitive function in a subject can include: obtaining the methionine sulfoxide-rich polypeptide of an embodiment; and administration of the methionine sulfoxide-rich polypeptide to a subject sufficient for immunizing the subject against the methionine sulfoxide-containing proteins.

A method of increasing clearance of methionine sulfoxide-containing proteins from a subject can include: obtaining the methionine sulfoxide-rich polypeptide of an embodiment; and administration of the methionine sulfoxide-rich polypeptide to a subject sufficient for immunizing the subject against the methionine sulfoxide-containing proteins.

In some embodiments, the methionine sulfoxide-containing proteins includes an Apo-J having an oxidized methionine residue.

In some embodiments, production of new Apo-J is induced due to clearance of Apo-J having an oxidized methionine residue.

In some embodiments, the MetO-rich polypeptide causes production of an anti-MetO antibody. In some embodiments, the anti-MetO antibody recognizes a MetO-containing protein in the subject for immunization thereof. In some embodiments, the immunization inhibits accumulation of MetO-containing proteins in the subject, such as in the brain or blood.

In some embodiments, the MetO-rich polypeptide provides immunization such that the subject's immune system can inhibit oxidative attack on the subject.

In some embodiments, the methods achieve improvement in short term memory and/or long-term memory. Such improvement can be before or during a neurodegenerative disease state. This allows for the methods to be used as a prophylactic before onset of symptoms or disease state, such that a subject can have improvement in memory without the disease state being present.

Amyloid refers to the abnormal fibrous, extracellular, proteinaceous deposits found in organs and tissues. Amyloid is insoluble and is structurally dominated by β-sheet structure. Unlike other fibrous proteins it does not commonly have a structural, supportive or motility role but is associated with the pathology seen in a range of diseases known as the amyloidoses. These amyloid-related diseases that can be treated with the MetO antigen immunization can include Alzheimer's, the spongiform encephalopathies and type II diabetes, all of which are progressive disorders with associated high morbidity and mortality. Accordingly, the MetO antigen can be used in immunization for treating or preventing reactive oxygen related diseases (e.g aging and age-associated diseases) including amyloid diseases, like Alzheimer's disease, Diabetes type 2, Atrial amyloidosis, Primary systemic amyloidosis, Senile systemic amyloidosis, Haemodialysis-related amyloidosis, Hereditary nonneuropathic systemic amyloidosis, Type II diabetes, Injection-localized amyloidosis, Secondary systemic amyloidosis, Hereditary cerebral amyloid angiopathy, Finnish hereditary systemic amyloidosis, Familial amyloid polyneuropathy I or II, Ageing pituitary, prolactinomas, Familial amyloidosis, British familial dementia, and the spongiform encephalopathies (e.g., Mad cow disease), or others.

### EXPERIMENTAL

The studies were performed by testing the MetO antigen for immunizing a well characterized AD-mice model (APP/PS1), in which mice were treated with either the MetO antigen plus adjuvant or adjuvant only at 14, 16, and 18 weeks of age. The adjuvant, Alum (Sigma-Aldrich) was mixed at 1:1 ratio (v/v) either with PBS (control group, n=9) or the MetO antigen moiety (50 μg) (experimental group, n=9). When the mice reached 40 weeks of age they were tested for their bio-behavioral performance using Y-maze test (measuring short memory capabilities) and Morris Water Maze (MWM) (measuring long and short memory and learning capabilities). Overall, the acquired data showed that the short and long memory capabilities have improved by −50-60%, with statistical significance, in the MetO antigen-immunized mice compared to controls. Also, the MWM test has showed alleviation of learning deficits observed in the AD-mice in the MetO antigen immunized group, in comparison to the control group. In addition, the MetO antigen-immunized mice demonstrated a-30% statistically significant reduction in their beta-amyloid level in blood-plasma compared to controls (the amyloid 1-42 (Aβ42) was detected using ELISA kit purchased from Thermo-Fisher Scientific).

The studies immunized transgenic AD (APP/PS1) mice at four months of age with a recombinant MetO-rich protein (e.g., MetO antigen) from Zea mays in the presence of adjuvant (Alum). This treatment induces the production of an anti-MetO antibody that in-turn reduces the levels of blood-plasma MetO-proteins. At ten months of age and compared to the control mouse (Alum-injected only), the MetO antigen injected mice exhibited the following significant phenotypes compared to controls: 1) better short and long memory capabilities, as judged by behavioral assays; 2) reduced Aβ levels in both blood-plasma and brain samples; 3) reduced Aβ burden in hippocampal and cortical regions; 4) reduced MetO accumulations in astrocytes of the hippocampal and cortical regions; 5) reduced levels of activated microglia (e.g., through monitoring the levels of the Iba1 protein marker); 6) and elevated antioxidant capabilities (e.g., through enhanced nuclear localization of the transcription factor Nrf2) in the same brain regions. The data strongly support the use of this MetO antigen for active immunization as a treatment for humans demonstrating symptoms of AD, as well as a prophylactic for delaying or preventing the onset the neurodegenerative disease or symptoms thereof.

### Materials and Methods

### Antibodies

Anti-Aβ₄₂ antibody (6E10) was purchased from BioLegend (San Diego, CA), which is a monoclonal antibody for the detection of amyloid plaque burden in brain sections of APP/PS1 mice at 10 months of age, when most, if not all, of the human APP is cleaved into Aβ. Anti-Iba1 and anti-Nrf2 antibodies were purchased from GeneTex (Irvine, CA). Anti-mouse/rabbit IgG HRP-conjugated antibody was purchased from Santa-Cruz Biotechnology (Santa Cruz, CA). Anti-rabbit/mouse fluorescent-labeled antibody was purchased from Thermo Fisher Scientific (Wathham. MA). Rabbit anti-MetO antibody was created and used according to our in-house procedures (20).

### MetO-Rich Protein (MetO Antigen) Production

The recombinant MetO-rich protein was produced similarly to the previous 6His-tagged MetO-rich protein (20), except of using 6His-Maltose Binding Protein (MBP) as the fusion protein instead of only the 6His-tag adduct. This change dramatically enhanced the production and purification yield of the recombinant protein. Codon optimized DNA of the Zea mays Met-rich protein (DZS18 protein) was synthesized from Integrated DNA technologies (Coralville, Iowa) and was subcloned into the pTBMalE plasmid (21). The resulting 6His-MBP-DZS18 construct was confirmed by DNA Sanger sequencing. Competent bacterial cells (BL21 (DE3)) pRARE were transformed with the 6His-MBP-DZS18 construct and positive clones were selected on agar plate supplement with 100 μg/ml ampicillin and 34 μg/ml of chloramphenicol. Protein was induced at log phase in LB media+antibiotic with 0.4 mM IPTG for 3 hours at 37° C. Thereafter, the cells were pelleted by centrifugation and frozen at −80° C. After the freeze/thaw cycle, cells were disrupted by sonication on ice. Following high-speed centrifugation (10,000×g) the supernatant was collected and the protein was purified through Ni affinity chromatography.

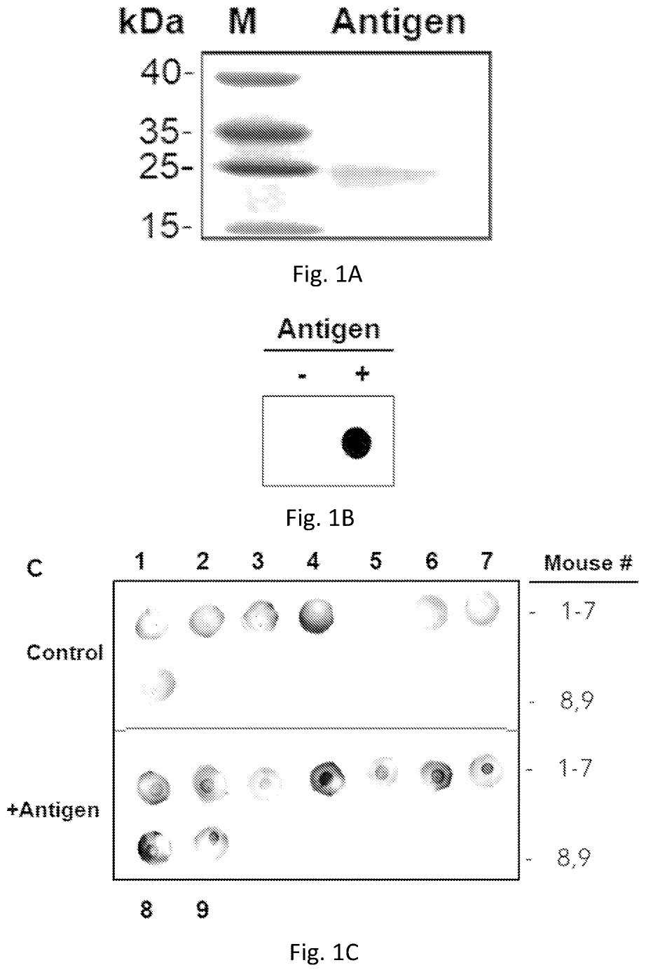

Briefly, the overexpressed 6His-MBP-DZS18 protein (596 aa, 65,833 DA) was purified to homogeneity from the supernatant by affinity purification using Ni²⁺-NTA resin (Qiagen, Germantown, MD), according to the manufacturer's protocol. The final buffer composition of the purified 6His-MBP-DZS18 protein was: 50 mM Tris-HCl, 0.5 M NaCl, and 500 mM imidazole. Then, the protein was digested with trypsin (trypsin: 6His-MBP-DZS18=1:10 (w/w) overnight at 37° C. The major resulting protein had 202 AA with a 22,629 Da mass and the minor one (following one more cleavage) had 163 AA with an 18,189 Da mass. The digested protein material was purified on SEC75 with a mobile phase buffer containing PBS+1M guanidine hydrochloride and the resulting peak protein was the major protein that was purified to homogeneity. Then, the protein was concentrated with a 10,000 DA cut-off centricon to remove any small remaining peptides. The final concentration of the protein was 1 mg/mL, and its purity was confirmed by SDS-gel-electrophoresis followed by Coomassie brilliant blue (Thermo-Fisher Scientific) staining (FIG. 1A; the limited protein staining level is due to the relative low presence of basic amino acids in the Met-rich protein sequence). Thereafter, the concentrated protein was then oxidized overnight at room temperature by adding to the protein mixture 200 mM of H₂O₂. The H₂O₂ decomposes with time and the protein was kept at −80° C. until use. Following MALDI-TOP analysis, the molecular mass of the purified oxidized protein was a 23,330 Da mass (predicted 202 AA with a 22,629 Da mass), indicating that 44 out of the protein's 48 Met residues were oxidized. To validate the immunogenicity of the MetO-rich protein, the protein was injected to a rabbit to create poly-clonal anti-MetO antibody as previously described (20). Indeed, the resulting antibody showed specificity towards Met-oxidized proteins (FIG. 1B), similarly to the previously described production of the anti-MetO antibody, using 6His-tagged MetO-rich protein as the antigen (20).

For the production of the MetO antigen, a person having ordinary skill in the art (“POSA”) would be able to use other fusion proteins, such as Glutathione-S-transferase (GST), Small Ubiquitin-like Modifier (SUMO), GB1 (B1 domain of streptococcus protein G) and other fusion proteins that have been widely used to increase the solubility of a recombinant protein) to produce the antigen. As such, the maltose binding protein (MBP) may be considered to be a solubility promoter, which can also include GST, SUMO, GB1 and others. The solubility promoter can be separated from the Met-rich polypeptide by a trypsin-site, which is cleaved by trypsin. This allows using trypsin to remove the solubility promoter polypeptide of the recombinant protein from the Met-rich polypeptide to facilitate production of the MetO antigen. It is equally conceivable that such a person can use other proteases in a similar fashion to generate the same antigen. Therefore, the solubility promoter polypeptide can be separated from the Met-rich polypeptide by a protease active site therebetween.

### Mice Immunization Procedure

A commonly used AD model mice (APP/PS1 (APPswe, PSEN1de9) 85Dbo, 18 female mice at age of ~1-2 months) were purchased from Jackson's lab and were immunized with the MetO-rich protein (MetO antigen). The mice were divided into two groups in which one was injected with the vehicle only, i.e., Alum adjuvant (Sigma-Aldrich) (control group) and one with the vehicle and MetO antigen (experimental group). Specifically, at ~3-4 months of age, the control group (9 females) was injected with Alum adjuvant only, while the experimental group (9 females) was injected with Alum plus the antigen (50 μg/mouse). Accordingly, two additional booster injections were performed in the following month (two-week intervals). The first injection was at 16 weeks; the second injection at 18 weeks; the third injection at 20 weeks; bio-behavioral testing at 40 weeks; and euthanasia and post-mortem analysis on blood and brain tissues at 42 weeks.

### Production of Anti-MetO Antibody in Rabbit and Mice

The MetO antigen (FIG. 1A) was injected into a rabbit to reproduce and validate its ability to create anti-MetO antibody. Indeed, only antigen-immunized rabbit showed a strong reaction against the antigen, following a dot-blot analysis (FIG. 1B). The dot-blot analysis is the preferred method for monitoring the anti-serum against MetO since the MetO antigen structure is closely mimicking its injected form (in an SDS-gel electrophoresis-western blot analysis the antigen is further modified by SDS). At 4 months of age, the APP/PS1 mice were injected with either the adjuvant only (control) or with the mixture of the adjuvant and antigen (experimental). An additional two boosts of injection were given in the following month in 2-week intervals. At 10 months of age, all the antigen-injected mice showed positive reactions against the antigen, while all the mice of the control group exhibited negative reactions (FIG. 1C). These results confirm that all the MetO antigen-injected mice were successfully immunized against the MetO antigen and obtained significant levels of anti-MetO antibody in sera throughout their life up to their euthanasia at 10 months of age.

FIGS. 1A-1C show that the MetO antigen-injected mice produce anti-MetO antibody against MetO-rich protein in blood-plasma. FIG. 1A shows the MetO-rich protein (MetO antigen) was produced and purified according to the procedures described herein. The purified MetO antigen was separated on SDS-gel-electrophoresis that was stained with Coomassie brilliant blue (the limited protein staining level is due to the relatively low presence of basic amino acids in the Met-rich protein sequence). M shows molecular mass markers; and kDa are the molecular mass numbers. FIG. 1B shows a dot-blot analysis using the MetO-rich protein as the loaded protein (MetO antigen) and rabbit anti-MetO antibody (1:1000 dilution) as the primary antibody. FIG. 1C shows a dot-blot analysis using the MetO-rich protein, as the loaded protein (MetO antigen), probed with plasma moiety (used as the primary antibody, 1:100 dilution, 1 h incubation time) from 10-month-old mice. Only the antigen-injected mice showed positive reactions with the antigen (a round black reaction in the middle of the blot), while the controls were all negative. Numbers 5 and 9 of the control blots had almost no background reactions.

### Y-Maze

The Y-maze test is a simple and common maze used to assess spatial working memory in mice by measuring their willingness to explore new environments (22). Mice were placed into the center of the 3-armed Y-maze, with one arm obstructed, and allowed to freely explore the two non-obstructed arms for 5 min. This served as a familiarizing training trial. Thirty-minutes later, mice were reintroduced into the Y-maze for a 5-min period, and in this trial, the full arena was accessible. Total arm entries as well as time spent in the novel and familiar arms were recorded. An extended period in the novel arm beyond chance is indicative of sustained cognition and short-term spatial memory, as the animals must remember which arm was not accessible during the training trial and thus a novel environment to explore.

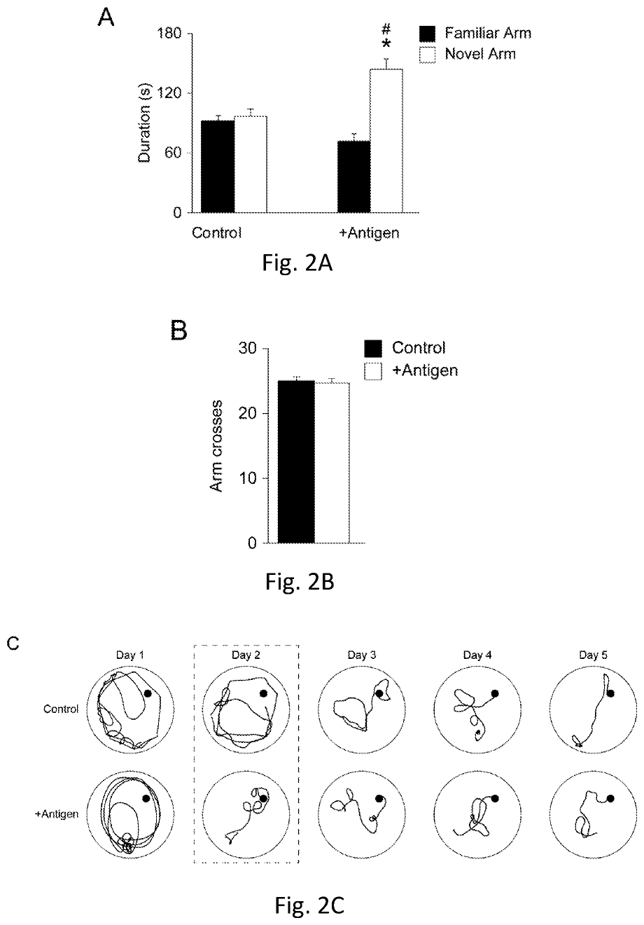

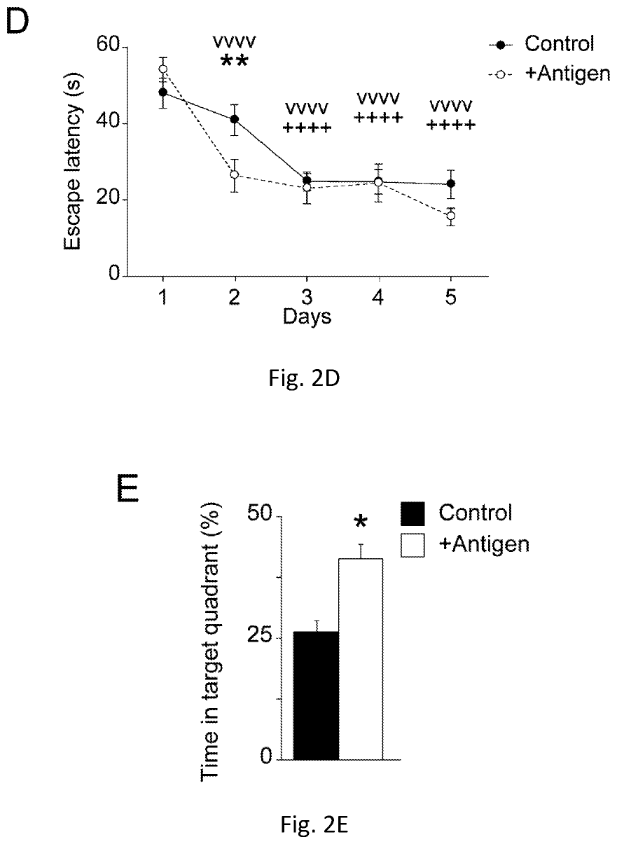

Cognitive impairment has been validated for APP/PS1 mice as early as 9 months of age. This includes short- and long-term spatial memory deficiencies. Thus, the control and antigen-immunized mice were studied in both Y-maze and Morris water maze tests at 10 months of age. In a Y-Maze task, the controls averaged about 103 s in the novel arm, which is right at the level of chance for a 300-s or 5-min test. MetO antigen-immunized mice spent significantly more time exploring a novel arm compared to the controls (One-way ANOVA, F (1,16)=9.022, p=0.008; FIG. 2A). Total arm crosses did not appear to be statistically different between groups (FIG. 2B), so there was no effect of treatment on locomotor behavior. Thus, antigen-immunized mice had improved short-term spatial memory and a novel arm preference, while the control mice failed to differentiate between familiar and novel arms.

### Morris Water Maze (MWM)

MWM is one of the most common behavioral tests used to determine hippocampal spatial short- and long-term memory deficits, particularly those observed in AD mouse models. MWM test was performed a week after the completion of the YMSA test (22). The training paradigm for the hidden platform version of the MWM consisted of 4 trials each day (with an interval of 15 min) for five consecutive days. The capacity of mice to retrieve and retain the learned location of the platform was assessed during a probe trial, which was carried out 24 h after the completion of training on the fifth day.

Over a 5-day training period, all animals eventually reduced their escape latency time (One-way ANOVA, F (4,64)=29.073, p<0.001; FIGS. 2C,D). However, MetO antigen-immunized mice improved their escape latency faster than the controls, dropping time significantly by the second day of training, while it required repeated days of training for the controls to reduce escape time (One-way ANOVA, F (4,64)=2.942, p=0.027). Furthermore, during the probe day (when the platform was removed from the MWM), the control mice spent 26.7% of the total time swimming in the target quadrant, indicating a failure to form long-term memory of the platform location (as 25% was the predicted chance percentage of time swimming in any given quadrant) (FIG. 2E). This failure in long-term spatial memory replicates published work in APP/PS1 mice at this age (23). Interestingly, the antigen-immunized females spent significantly more time in the target quadrant than the controls (two-tailed t-test t (16)=2.615, p=0.019; FIG. 2E). Together with the Y-maze data, it appears the antigen vaccination significantly slowed the age-dependent cognitive decline observed in short-term and long-term memory, typically demonstrated in APP/PS1 mice.

FIGS. 2A-2E show that the vaccinated APP/PS1 mice had improved short-term and long-term spatial memory. FIG. 2A shows that the MetO antigen-immunized APP/PS1 mice have a novel arm preference in a forced Y-maze test but control APP/PS1 do not, displaying improved short-term memory after vaccination. FIG. 2B shows that arm crossing is not affected by vaccination, indicating age-typical locomotion. FIG. 2C shows the MWM analyses. These are representative schematics of swimming patterns performed by mice over the 5-day training period of the MWM. These swimming patterns illustrate that MetO antigen-immunized APP/PS1 mice improve their escape latency after a single day of training, while control mice require repeated training days to improve in this task (as reflected in Day 2). FIG. 2D shows the statistical presentation of the significant effect on the antigen-immunized mice's reduced escape latency during the second day of training, while the control mice do not improve in this task until day 3. This demonstrates an improved learning for this long-term spatial memory task in MetO antigen-immunized mice. FIG. 2E shows that MetO Antigen-immunized mice also spend more time swimming in the target quadrant (after the platform is removed from the pool) compared to controls, who are only per-forming at chance. Standard deviation is shown for each group of mice. Data were acquired from all tested mice (n=9 per each group of mice). Statistical significances were assessed for the acquired data using one-way analysis of variance (ANOVA) with post hoc Tukey's test: #p<0.05 familiar vs. novel arm within condition; *p<0.05, **p<0.01 vs. control; vvvv p<0.0001 vs. day 1 training for antigen-immunized mice; ++++p<0.0001 vs. day 1 training for control mice. Monitoring total Aβ42 levels in mouse blood-plasma and brain

At 10 months of age, the mice were perfused with PBS, and samples of their blood was collected, and brains were extracted. The soluble blood-plasma was separated and collected by centrifugation for 15 min at 2000×g in the presence of 1 mM EDTA. Soluble proteins from one hemisphere of each brain were obtained following guanidine-HCl extraction as described by the ELIZA kit protocol for Aβ₄₂ (Thermo-Fisher Scientific). The corresponding levels of Aβ₄₂ in blood-plasma and total brain of each mouse was measured according to the protocols provided by the same ELIZA kit.

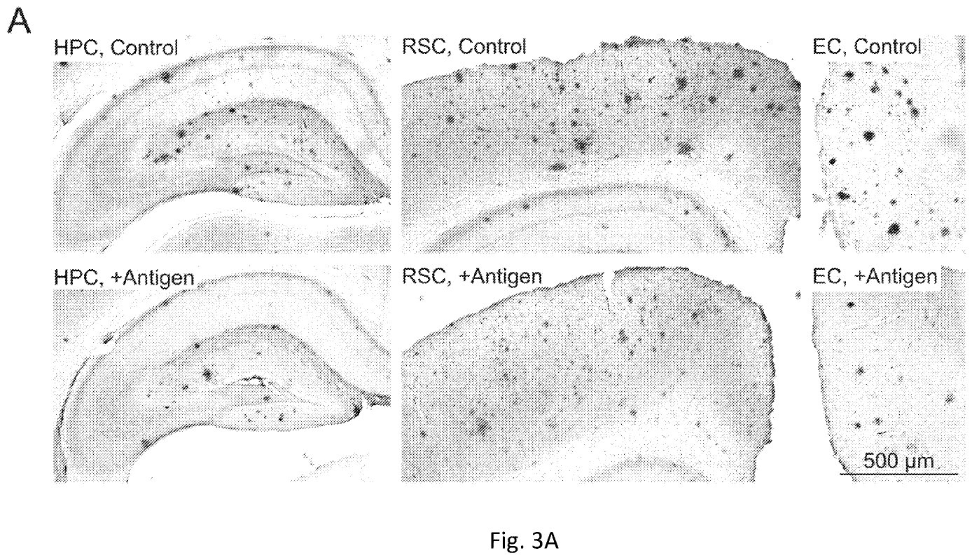

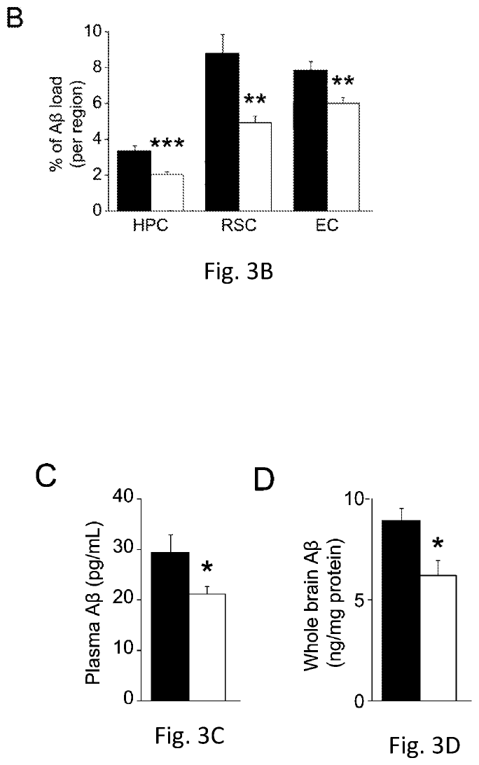

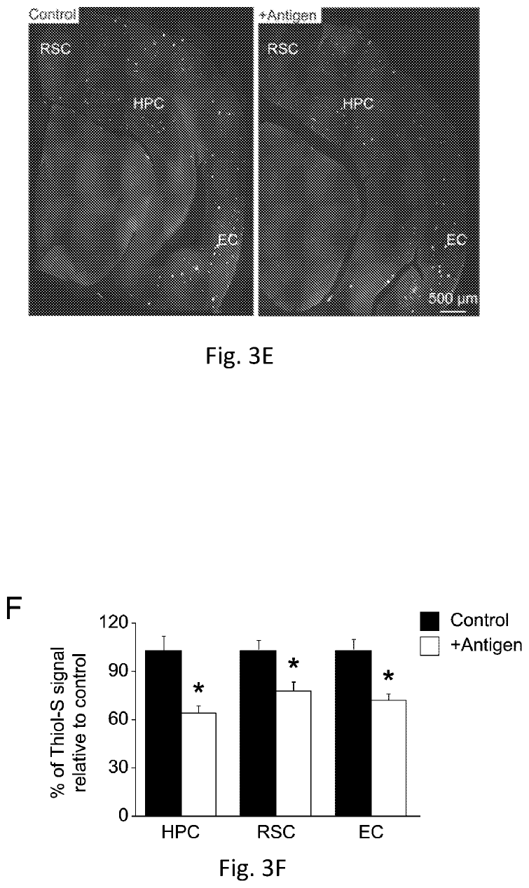

At 10 months of age, the levels of Aβ₄₂ in blood-plasma and whole-brain extracts of postmortem mice were analyzed. Compared to the control group, the antigen-immunized mice showed an average of 31% and 28% decline in the level of blood-plasma and whole-brain Aβ₄₂, respectively (FIGS. 3 C,D, two-tailed t-test, t₍₁₁₎=2.495 (plasma), p=0.02; t₍₁₁₎=2.313 (brain), p=0.04). Immunohistochemistry analyses of the hippocampal and cortical regions of the brain (hippocampus (HPC); retrosplenial cortex (RSC) and entorhinal cortex (EC)) demonstrated similar results (FIGS. 3A,B). Specifically, compared to controls, the antigen-immunized mouse brain regions exhibited an average decline in the levels of Aβ42 as follows: 38% in the HPC, 42% in the RSC, and 29% in the EC (FIG. 3B, two-tailed t-test, ((13)=6.432, p=0.0002 for HPC; two-tailed t-test, f (12)=3.883, p=0.003 for RSC, and 3. 546, p=0.002 for EC). These data were corroborated by Thioflavin-S staining that enables the detection of β-pleated sheet conformation of amyloid (although this staining is less specific relative to the immunohistochemistry analysis). Accordingly, compared to the controls, the antigen-immunized mouse brain regions exhibited an average decline in the levels of Thioflavin-S staining as follows: 34% in the HPC, 20% in the RSC, and 26% in the EC (FIG. 3E,F, two-tailed t-test, t₍₁₉₎=2.488, p=0.04 for HPC; t₍₁₉₎=2.107, p=0.02 for RSC; t₍₁₉₎=2.405, p=0.02 for EC). Overall, these observations show that the levels of Aβ in both blood-plasma and brains of the antigen-injected mice were significantly reduced compared to the control mice.

FIGS. 3A-3F show the reduction of Aβ₄₂ in blood-plasma and brain, and reduction of amyloid plaque burden in brains of antigen-injected mice. FIG. 3A shows the hemisphere brains of post-mortem mice (control and anti-gen-immunized) that were dissected, and immunohistochemistry analyses were performed to detect Aβ₄₂. The data shows a decline in the levels of Aβ₄₂ in the three brain regions (HPC, hippocampus; RSC, retrosplenial cortex, and EC, entorhinal cortex) of the antigen-immunized as compared with the control mice. FIG. 3B shows the quantification of the data presented in FIG. 3A using the NIH-Image J program. Quantification of Aβ₄₂ in blood-plasma is shown in FIG. 3C and post-mortem brain extracts are shown in FIG. 3D of antigen-injected and control mice using ELISA kit according to the procedure described. FIG. 3E shows the thioflavin-S staining of the post-mortem mouse brains. A lower plaque-burden was observed in the antigen-injected versus control mice. FIG. 3F shows the quantification of the data depicted in FIG. 3E using the NIH Image-J program. In all of the graphs, black bars represent control and white bars represent antigen-injected mice. Standard deviation is shown for each averaged bar. Data were acquired from all tested mice for panels C and D (n=9 per each group of mice) and five mice per each tested group of mice for FIGS. 3B and 3F. Statistical analyses were performed using two-tailed student t-test with the following p values: *, p<0.05; **, p<0.01, and ***, p<0.001.

### Immunohistochemistry and Staining of Mouse Brains' Slices

The complementary hemisphere of each postmortem PBS-perfused brain was fixed in 4% paraformaldehyde in PBS at 4° C., and then flash frozen by exposure to dry ice. Coronal sections (30 μm) were cut using a cryotome and transferred to gelatin-coated glass slides. For the detection of β-pleated sheet conformation of amyloid, sections were stained with Thioflavin-S dye solution (1%), rinsed in water, dehydrated through graded ethanol, cleared with xylene, and finally be mounted with permount (24). For immunohistochemistry staining, sections were blocked with 3% (w/v) gelatin in PBS (1 h at 37° C.), treated with 0.1% Triton X-100 in PBS (15 min at 23° C.), and reacted (overnight at 4° C. plus 1 h at 23° C.) with the selected primary antibody. After rinsing in PBS, the sections were incubated (2 hours at room-temp) with either primary antibody-matched fluorescent dye-labeled secondary antibodies (Alexa 568 goat anti-rabbit or Alexa 488 goat anti-mouse; Thermo Fisher Scientific) or HRP-labeled secondary goat anti-mouse/rabbit antibody (Santa-Cruz Biotechnology). Then, the sections were rinsed with PBS and followed by Tris-EDTA buffer, mounted, viewed, and analyzed using a light microscope. Quantification of the emitted signal per brain section was achieved by calculating the mean signal coverage per same brain area using the NIH Image-J program or manually counting the nuclear Nrf2 signal per same-size area in the three selected brain regions (HPC, RSC, and EC, n=4 brain slices per mouse).

### Levels of MetO-Proteins in Brain Cells

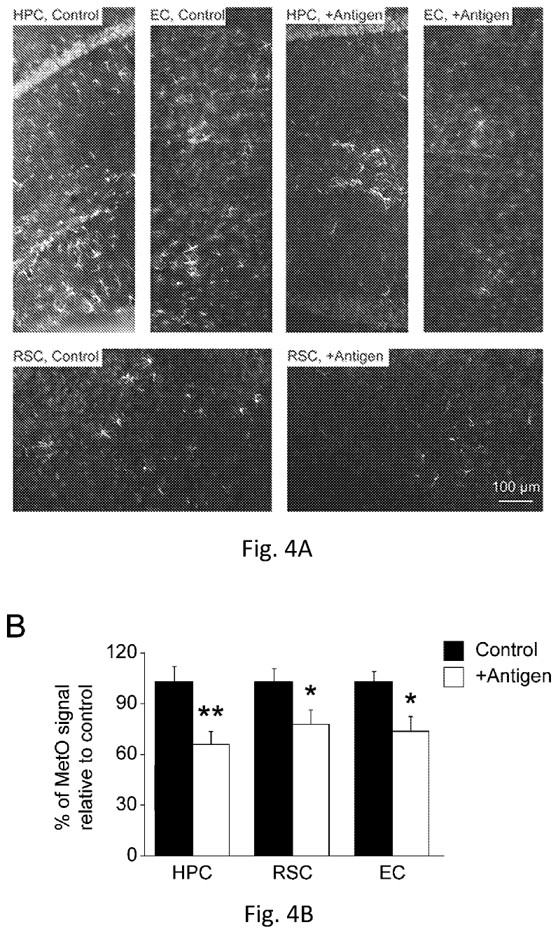

The vaccination-mediated alleviation of oxidative stress may be evident by the existence of lower levels of MetO-proteins in the brain. Indeed, as shown in FIGS. 4A-4B, the control mice had higher levels of MetO-proteins in brain astrocytes, compared to the antigen-injected mice. Specifically, compared to controls, the antigen-immunized mouse brain regions exhibited an average decline in the levels of MetO-proteins as follows: 36% in the HPC, 25% in the RSC, and 23% in the EC (FIG. 4A,B, two-tailed t-test, t₍₁₇₎=3.142, p=0.006 for HPC); two-tailed t-test, t₍₁₈₎=2.613, p=0.04 for RSC; t-test, t₍₁₈₎=2.192, p=0.02 for EC).

FIGS. 4A-4B show the reduction of MetO-protein levels in brain astrocytes in antigen-injected mice. FIG. 4A shows hemisphere brains of post-mortem mice (control and antigen-immunized) were dissected, and immunohistochemistry analyses were performed to detect MetO-proteins according to the procedure described. The data shows a decline in the levels of MetO-proteins in the three brain regions (HPC, hippocampus; RSC, retrosplenial cortex, and EC, entorhinal cortex) of the antigen-immunized compared with control mice. FIG. 4B shows the quantification of the data presented in FIG. 4A using the NIH-Image J program. Black bars represent control and white bars represent antigen-injected mice. Standard deviation is shown for each averaged bar. Data were acquired for five mice per tested group of mice. Statistical analyses were performed using two-tailed student t-test with the following p values: *, p<0.05, and **, p<0.01.

### Indices of Gene Regulation in Brain Cells

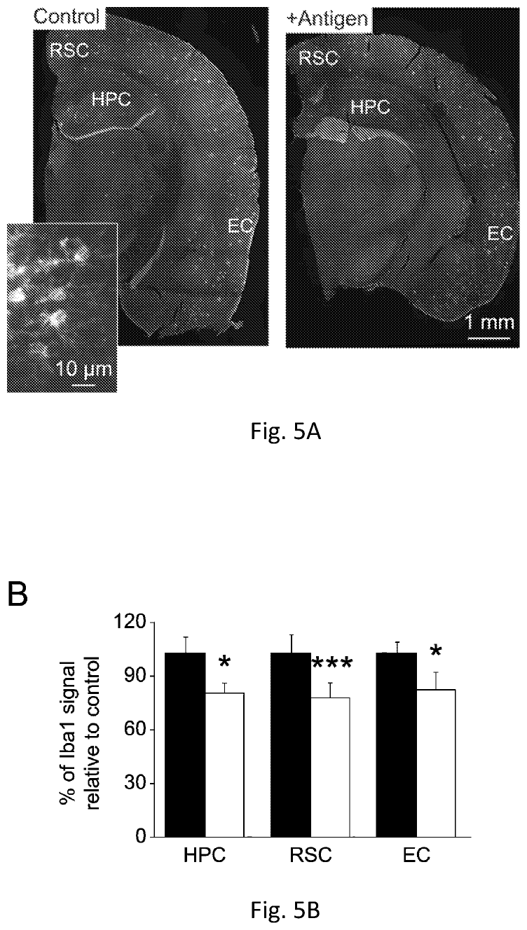

Activation of microglia assist in the clearance of Aβ deposits in brain and thus their activation level corelates with Aβ level. Therefore, a reduction of the level of activated microglia upon antigen vaccination is expected since lower Aβ deposits were observed in the antigen-vaccinated mice (see FIGS. 3A-3F). Accordingly, compared to control mice, all the tested brain regions of the antigen-vaccinated mice showed lower levels of activated microglia (as judged by the levels of the activated microglia marker protein Iba1) (FIGS. 5A-5B). Specifically, compared to controls, the antigen-immunized mouse brain regions exhibited an average decline in the levels of Iba1-protein as follows: 25% in the HPC, 24% in the RSC, and 26% in the EC (FIGS. 5A-5B, two-tailed t-test, P<0.05 (HPC and EC), 12 deg of freedom; P<0.01 (RSC) 15 deg of freedom).

FIGS. 5A-5B show the reduced activation of microglia in MetO antigen-injected mice. FIG. 5A shows the hemisphere brains of post-mortem mice (control and antigen-immunized) were dissected, and immunohistochemistry analyses were performed to detect Iba1 according to the procedure described. The data show a decline in the levels of Iba1 of neuronal cells in the three brain regions (HPC, hippocampus; RSC, retrosplenial cortex, and EC, entorhinal cortex) of the antigen-immunized compared with control mice. FIG. 5B shows the quantification of the data presented in FIG. 5A using the NIH-Image J program. Black bars represent control and white bars MetO antigen-injected mice, respectively. Standard deviation is shown for each averaged bar. Data were acquired from five mice per tested group of mice. Statistical analyses were performed using two-tailed student t-test with the following p values: *, p<0.05; ***, p<0.001.

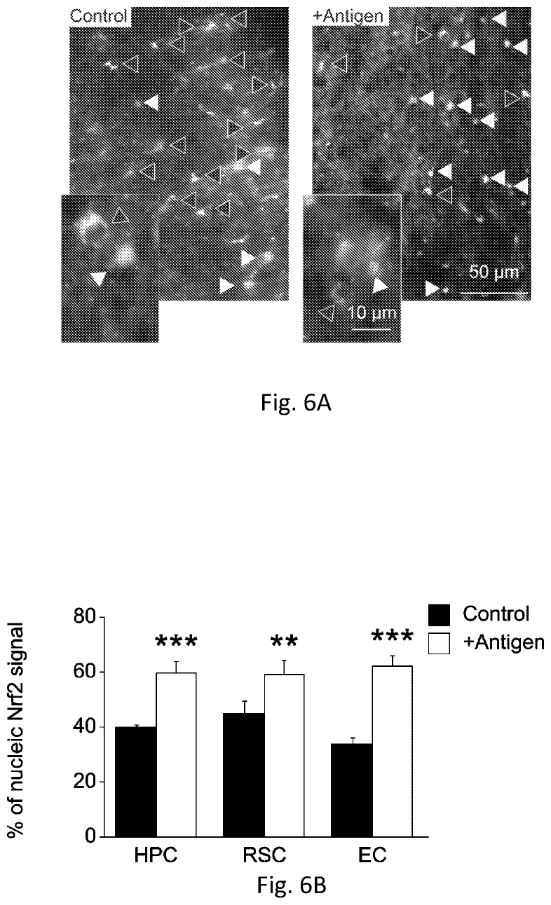

An important transcription factor that regulates the expression of several antioxidant genes is Nrf2 (25). This function of Nrf2 is mediated through its translocation to the nucleus from the cytoplasm, causing an enhanced transcription of several genes, including antioxidant genes (25). Hence, a relatively high nucleic localization of Nrf2 is beneficial in providing a strong cellular antioxidant protection. Interestingly, compared to the control mice, the antigen-immunized mice demonstrated a higher rate of nuclear localization of Nrf2 mainly in astrocytes (FIG. 6A, B), suggesting a positive effect of the antigen on Nrf2 function through a yet-to-be-discovered mechanism. Specifically, compared to the controls, the antigen-immunized mouse brain regions exhibited an average rate increase of Nrf2-nuclear localization as follows: 59% vs. 39% in the HPC, 57% vs. 44% in the RSC, and 60% vs. 31% in the EC (two-tailed t-test, t₍₁₃₎=7.882, p=0.0004 for HPC; t-test two-tailed t-test, t₍₁₇₎=3.135, p=0.006 for RSC; t-test, t₍₁₃₎=7.619, p=0.0005 for EC). These data correlate with the observation of lower MetO-protein levels in astrocytes of the antigen-immunized mice (FIGS. 3A-3F).

FIGS. 6A-6B show increased nuclear localization of Nrf2 in MetO antigen-injected mice. FIG. 6A shows hemisphere brains of post-mortem mice (control and antigen-immunized) were dissected, and immunohistochemistry analyses were performed to detect Nrf2 according to the procedure described. The data shows an increased nuclear localization within astrocytes of Nrf2 in a representative image of the EC region of the antigen-immunized compared to the control mice. A similar pattern of Nrf2 localization ratio between the two mouse groups was observed for the HPC and RSC regions (images are not shown). The white triangle symbols point to cells harboring nuclear Nrf2 and the black-filled triangle symbols point to cells harboring cytosolic Nrf2. FIG. 6B shows quantification of the data presented in FIG. 6A by manually counting the nuclear Nrf2 signal per same-size area in the three selected brain regions. Black bars represent control and white bars represent MetO antigen-injected mice, respectively. Standard deviation is shown for each averaged bar. Data were acquired from five mice per tested group of mice. Statistical analyses were performed using two-tailed student t-test with the following p values: ***, p<0.001 and **, p<0.01.

### Apolipoprotein J (Apo-J, Clusterin) and Alzheimer's Disease (AD)

Apo-J is a secreted protein with chaperon functions that is highly expressed in brain and plasma. This protein can bind to non-native proteins, like forms of beta-amyloid peptides, and thereby prevent their extracellular aggregation. Thus, this Apo-J function is considered beneficial in preventing the onset or progression of AD when the Apo-J function is intact. However, compromised Apo-J function may promote the onset or progression of AD.

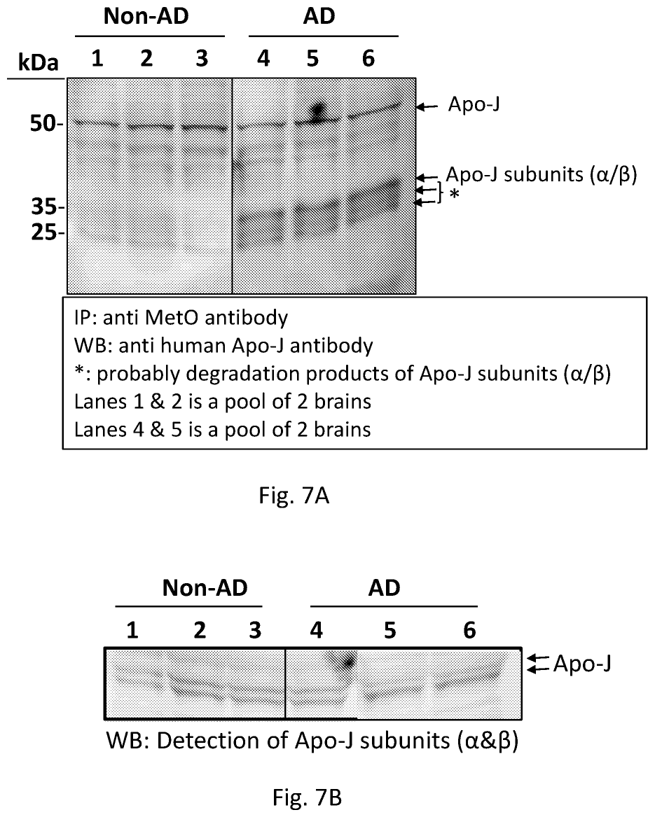

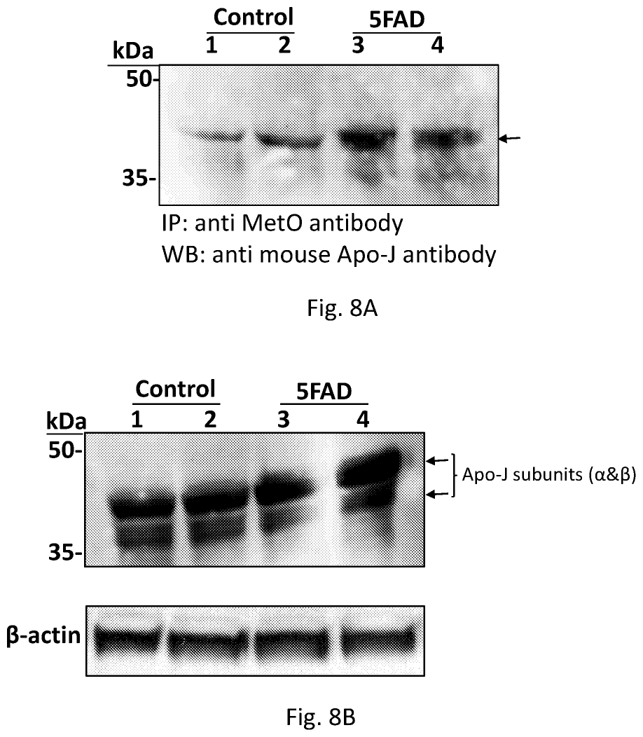

The normal percentage of Met residues in a typical protein is ~2%. The percentage of Met residues of Apo-J is ~3.5%, a feature that enhances the chances for its methionine oxidation. Accordingly, it has now been discovered that the level of MetO is increased in postmortem human AD brains compared to controls (FIGS. 7A-7B). These data were corroborated in postmortem AD mouse model brains, compared to control brains, as shown in FIGS. 8A-8B. These findings suggest that the enhanced methionine oxidation of Apo-J in AD compromises its chaperon function against beta-amyloid aggregation, leading to AD progression. It is hypothesized that clearance of the MetO-Apo-J (through the active MetO-rich protein immunization) will foster de-novo production and secretion of non-oxidized Apo-J, thereby increasing the protective ability of Apo-J against the progression of AD.

FIGS. 7A-7B show detection of methionine-oxidized Apo-J in postmortem human brains. Postmortem human brain samples of late stages of AD (hippocampus region, n=5) and at late stages of non-AD brains (Parkinson disease (PD), hippocampus region, n=5) were incubated in PBS in the presence of 0.32M sucrose and protease inhibitors (Sigma-Aldrich) for 4 hours at 4° C. Equal amounts of the resulting extracellular protein moiety from each brain were subjected to immunoprecipitation (IP), using the rabbit anti-MetO antibody; followed by western-blot (WB) analysis, using a primary mouse anti-human Apo-J antibody (Proteintech). Lane 1, an average load of two brain protein extracts; lanes 2 & 3, loads of one extracted brain per each lane. The data shows that the level of MetO is increased in postmortem human AD brains compared to controls.

Western blot analysis was performed with extracellular brain protein extracts obtained for the brains described in FIG. 7A (using the same mouse anti-human Apo-J antibody). Equal amounts of protein extracts were loaded per each lane. Accordingly, the data show no significant difference in the expression of Apo-J between the AD and the Non-AD brains, as shown in FIG. 7B.

FIGS. 8A-8B show detection of methionine-oxidized Apo-J in postmortem mouse brains. With regard to FIG. 8A, postmortem mouse brain samples of late stages of AD (5FAD mouse model of AD and corresponding controls; n=5 per strain). Whole brains were extracted in PBS in the presence protease inhibitors (Sigma-Aldrich). The resulting extracted protein moiety contains both cellular and extracellular proteins in each brain. Equal amounts of protein extracts per brain were subjected to immunoprecipitation (IP), using the rabbit anti-MetO antibody; followed by western-blot (WB) analysis (FIG. 8A), using a primary goat anti-mouse Apo-J antibody (Proteintech). Lane 1 has an average load of two brain protein extracts; lane 2 has an average load of three brain protein extracted. kDa, molecular mass markers.

FIG. 8B shows Western blot analysis of secreted brain protein extracts was obtained for the brains described in FIG. 8A (using the same mouse anti-human Apo-J antibody). Accordingly, the data show no significant difference in the expression of Apo-J between the 5FAD and control brains. The 8-actin expression levels serve as loading controls (using anti-β-actin primary antibody, Thermo-Fisher Scientific). kDa, molecular mass markers.

### Vectors and Polypeptides

The DNA coding E. coli mMBP was inserted into pTBSG, which is derived from pMCGS7 (26) The plasmid was a generous gift from Dr. Mark I. Donnell from University of Wisconsin), resulting in a vector called pTBMalE according to our previous report (27). The pTBMalE vector results in a hybrid polypeptide of SEQ ID NO: 1.

SEQ ID NO: 1 is:
MHHHHHHSTS KIEEGKLVIW INGDKGYNGL AEVGKKFEKD TGIKVTVEHP
DKLEEKFPQV AATGDGPDII FWAHDRFGGY AQSGLLAEIT PDKAFQDKLY
PFTWDAVRYN GKLIAYPIAV EALSLIYNKD LLPNPPKTWE EIPALDKELK
AKGKSALMEN LQEPYFTWPL IAADGGYAFK YENGKYDIKD VGVDNAGAKA
GLTFLVDLIK NKHMNADTDY SIAEAAFNKG ETAMTINGPW AWSNIDTSKV
NYGVTVLPTF KGQPSKPFVG VLSAGINAAS PNKELAKEFL ENYLLTDEGL
EAVNKDKPLG AVALKSYEEE LAKDPRIAAT MENAQKGEIM PNIPQMSAFW
YAVRTAVINA ASGRQTVDEA LKDAQTGTEN LYFQSNAHIQ ALVTTTDAIG
YHEPWMQYCM KQQGVANLLA WPTLMLQQLL ASPLQQCQMP
MMMPGMMPPM TMMPMPSMMP SMVPTMMSPM TMASMMPPMM
MPSMISPMTM PSMMPSMIMP TMMSPMIMPS MMPPMMMPSM
VSPMMMPNMM TVPQCYSGSI SHIIQQQQLP FMESPTAMAI PPMFLQQPFV
GAAF

SEQ ID NO: 1: Number of amino acids: 574; Molecular weight: 63633.35.

In SEQ ID NO: 1, the underlined sequences indicate specific proteases cleave sites (1. 2. Tobacco etch virus protease (TEV) protease) inserted according to the protein sequence for common purification purposes (28). Not used in our studies.

After trypsin cleavage (cleaves at R and K residues), with the first cleavage site and major protein product, as shown with SEQ ID NO: 2.

SEQ ID NO: 2:
DAQTGTENLY FQSNAHIQAL VITTDAIGYH EPWMQYCMKQ QGVANLLAWP
TLMLQQLLAS PLQQCQMPMM MPGMMPPMTM MPMPSMMPSM
VPTMMSPMTM ASMMPPMMMP SMISPMTMPS MMPSMIMPTM MSPMIMPSMM
PPMMMPSMVS PMMMPNMMTV PQCYSGSISH IIQQQQLPFM FSPTAMAIPP
MFLQQPFVGA AF

SEQ ID NO: 2 (MetO-rich protein) includes: Number of amino acids: 202 and Molecular weight: 22628.77, and has the amino acid composition with the number of each represented amino acid and the percentage thereof in Table 1.

TABLE 1
Amino Acid Composition:

| Amino Acid | Number | Percentage |
|---|---|---|
| Ala (A) | 12 | 5.9% |
| Arg (R) | 0 | 0.0% |
| Asn (N) | 4 | 2.0% |
| Asp (D) | 2 | 1.0% |
| Cys (C) | 3 | 1.5% |
| Gln (Q) | 18 | 8.9% |
| Glu (E) | 2 | 1.0% |
| Gly (G) | 6 | 3.0% |
| His (H) | 3 | 1.5% |
| Ile (I) | 9 | 4.5% |
| Leu (L) | 11 | 5.4% |
| Lys (K) | 1 | 0.5% |
| Met (M) | 50 | 24.8% |
| Phe (F) | 6 | 3.0% |
| Pro (P) | 32 | 15.8% |
| Ser (S) | 18 | 8.9% |
| Thr (T) | 13 | 6.4% |
| Trp (W) | 2 | 1.0% |
| Tyr (Y) | 4 | 2.0% |
| Val (V) | 6 | 3.0% |

There is also a minor product from the second cleavage site, which is SEQ ID NO: 3.

SEQ ID NO 3:
QQGVANLLAW PTLMLQQLLA SPLQQCQMPM MMPGMMPPMT
MMPMPSMMPS MVPTMMSPMT MASMMPPMMM PSMISPMTMP
SMMPSMIMPT MMSPMIMPSM MPPMMMPSMV SPMMMPNMMT
VPQCYSGSIS HIIQQQQLPF MFSPTAMAIP PMFLQQPFVG
AAF

SEQ ID NO: 3 includes: Number of amino acids: 163 and Molecular weight: 18168.83 and has the amino acid composition with the number of each represented amino acid and the percentage thereof in Table 2.

TABLE 2
Amino Acid Composition:

| Amino Acid | Number | Percentage |
|---|---|---|
| Ala (A) | 8 | 4.9% |
| Arg (R) | 0 | 0.0% |
| Asn (N) | 2 | 1.2% |
| Asp (D) | 0 | 0.0% |
| Cys (C) | 2 | 1.2% |
| Gln (Q) | 14 | 8.6% |
| Glu (E) | 0 | 0.0% |
| Gly (G) | 4 | 2.5% |
| His (H) | 1 | 0.6% |
| Ile (I) | 7 | 4.3% |
| Leu (L) | 9 | 5.5% |
| Lys (K) | 0 | 0.0% |
| Met (M) | 48 | 29.4% |
| Phe (F) | 5 | 3.1% |
| Pro (P) | 31 | 19.0% |
| Ser (S) | 17 | 10.4% |
| Thr (T) | 8 | 4.9% |
| Trp (W) | 1 | 0.6% |
| Tyr (Y) | 1 | 0.6% |
| Val (V) | 5 | 3.1% |

An example of a 6His-tagged Met-rich protein sequence (PQE 30 expression vector) is SEQ ID NO 4:

MHHHHHHSTS VDLGTENLYF QSNAMAAKMF ALFALLALCA
TATSATHIQA LVTTTDAIGY HEPWMQYCMK QQGVANLLAW
PTLMLQQLLA SPLQQCQMPM MMPGMMPPMT MMPMPSMMPS
MVPTMMSPMT MASMMPPMMM PSMISPMTMP SMMPSMIMPT
MMSPMIMPSM MPPMMMPSMV SPMMMPNMMTV PQCYSGSISH
IIQQQQLPFMF SPTAMAIPPM FLQQPFVGAA F

SEQ ID NO: 4 includes: Number of amino acids: 230 and Molecular weight: 24562.1, and the underlined sequence are specific TEV protease site for conventional removal of the 6His tag moiety (not used in our experiments).

### Protocol to Produce Methionine (Met)-Rich Protein (DZS18, Zea Mays)

### Making the Expression Plasmid Construct

A cDNA chip for the DZS18 sequence was obtained as a gblock and was amplified by PCR using the following primers:

(SEQ ID NO: 5)
5′-TACTTCCAATCCAATGCGcatatccaggcacttgtcac
(SEQ ID NO: 6)
3′-TTATCCACTTCCAATGctagaalgcagcaccaacaaag

The Maltose Binding Protein (MBP) expression plasmid was pTBMalE. In principle, any 6HIS-MBP commercially available expression vector can be used for the expression and purification of the DZS18 since the purified fusion protein (6HIS-MBP-DZS18) is digested with trypsin and all the created cleavage sites created in the pTBMalE are not relevant.

### Bacterial Expression of the Plasmid Construct and MBP-DZS18 Protein Purification

Competent bacterial cells (BL21 (DE3)) were transformed with the 6His-MBP-DZS18 construct and positive clones were selected on agar plate plus 50 μg/mL ampicillin. One clone was grown in LB media+antibiotic and induced with 1 mM IPTG for 3 hours. Thereafter, the cells were precipitated by centrifugation and their protein moiety was extracted by sonication at 4° C. Following high-speed centrifugation (10,000×g) the supernatant was collected and subjected to further purification steps. Briefly, the expressed fused 6HIS-MBP-DZS18 protein (596 aa, 65833 DA) was purified to homogeneity from the supernatant by affinity purification using Ni²⁺-NTA resin (Qiagen), according to the manufacturer's protocol. The final buffer composition of the 6His-MBP-DZS18 protein was: 50 mM Tris-HCl, 0.5 M NaCl. Then the protein was subjected to trypsin digestion as described below.

### Trypsin-Treatment of 6HIS-MBP-DZS18 Protein and Oxidation of the Resulting P18 Protein

The purified 6HIS-MBP-DZS18 protein was digested with trypsin (trypsin: 6HIS-M=1:10 (w/w) in 50 mM Tris-HCl, pH 8.0, for overnight at 37° C. The major resulting protein had 202 AA with a 22629 Da mass and the minor one (following one more cleavage) had 163 AA with an 18189 Da mass.

The digested protein material was purified on SEC75 with a mobile phase buffer containing PBS+1M guanidine hydrochloride and the resulting peak protein was the major protein that was purified to homogeneity. Then, the protein was concentrated with 3000 DA cut-off centricon and to remove any small remaining proteins. The concentrated protein was then oxidized overnight at room temperature by adding to the protein mixture 200 mM of H₂O₂. The H₂O₂ decomposes with time and the protein was kept at −80° C. until use.

Following MALDI-TOP analysis the molecular mass of the purified oxidized protein was a 23,330 Da mass (predicted 202 AA with a 22629 Da mass), indicating that 44 out of the protein's 48 Met residues were oxidized, which is the MetO-rich protein used as the antigen in the immunization strategies described herein.

The new 6His-MBP-Met antigen fusion protein production was about 1 mg per 1 liter compared with 1 mg of 6His-antigen (protein made in the first patent) per 500 liters of bacterial culture growth for the over expression bacterial strain.

Insert Gene: DZS18 Gene (cDNA) from Zea Maize

Another embodiment of a plasmid construct is provided that expresses the Met-rich protein (used to form the MetO antigen protein) using a commercial vector from New England Biolabs (pMal-6ct). The construct contains the whole reading frame of the corn protein, compared to the version that is truncated with several amino acids at its N-terminus. This change does not make any difference with respect to the final purified protein end product and its potential antigenicity when oxidized into the MetO antigen. The trypsin will digest the addition as well, which cleave into the Met-rich protein that can be oxidized into the MetO antigen. In some aspects, the whole reading frame of the antigen can be used This alternative construct is that the vector is commercially available, making it more suitable to reproduce the expression construct, in case one needs to recreate it from commercially available sources.

The cDNA sequence of the DZS18 gene is SEQ ID NO: 7.

SEQ ID NO: 7:
ATGGCAGCCAAGATGTTTGCATTGTTTGCGCTCCTAGCTCTTTGTG
CAACCGCCACTAGTGCTACCCATATCCAGGCACTTGTCACCACTA
CTGATGCCATTGGCTACCATGAACCATGGATGCAGTACTGCATGA
AGCAACAGGGGGTTGCCAACTTGTTAGCGTGGCCGACCCTGATGC
TGCAGCAACTGTTGGCCTCACCGCTTCAGCAGTGCCAGATGCCAA
TGATGATGCCGGGTATGATGCCACCGATGACGATGATGCCGATGC
CGAGTATGATGCCATCGATGGTGCCGACTATGATGTCACCAATGA
CGATGGCTAGTATGATGCCGCCGATGATGATGCCAAGCATGATTT
CACCAATGACGATGCCGAGTATGATGCCTTCGATGATAATGCCGA
CCATGATGTCACCAATGATTATGCCGAGTATGATGCCACCAATGA
TGATGCCGAGCATGGTGTCACCAATGATGATGCCAAACATGATGA
CAGTGCCACAATGTTACTCTGGTTCTATCTCACACATTATACAAC
AACAACAATTACCATTCATGTTCAGCCCCACAGCCATGGCGATCC
CACCCATGTTCTTACAGCAGCCCTTTGTTGGTGCTGCATTCTAG

After trypsin cleavage (cleaves at R and K residues), with the first cleavage site and major protein product, as shown with SEQ ID NO: 8. Thus, the corresponding Met-rich protein that can be oxidized into a MetO antigen is SEQ ID NO: 8.

SEQ ID NO: 8:
MAAKMFALFA LLALCATATS ATHIQALVTT TDAIGYHE PWMQYCMKQQ
GVANLLAWPT LMLQQLLASP LQQCQMPMMM PGMMPPMTMM
PMPSMMPSMV PTMMSPMTMAS MMPPMMMPSMI SPMTMPSMMP
SMIMPTMMSPM IMPSMMPPMMM PSMVSPMMMPN MMTVPQCYSGS
ISHIIQQQQLP FMFSPTAMAI PPMFLQQPFV GAAF

The forth amino acid in the sequence SEQ ID NO: 8 is “K”, which may be cleaved by trypsin. So, the final protein may start from the second “M” residue of the whole protein coding sequence, which is now shown in SEQ ID NO: 13.

SEQ ID NO: 13
MFALFA LLALCATATS ATHIQALVTT TDAIGYHE PWMQYCMKQQ
GVANLLAWPT LMLQQLLASP LQQCQMPMMM PGMMPPMTMM
PMPSMMPSMV PTMMSPMTMAS MMPPMMMPSMI SPMTMPSMMP
SMIMPTMMSPM IMPSMMPPMMM PSMVSPMMMPN MMTVPQCYSGS
ISHIIQQQQLP FMFSPTAMAI PPMFLQQPFV GAAF

The pMAL-c6T Vector for the expression of N-terminus 6His-Maltose-binding protein fusion was obtained from New England Biolabs as SEQ ID NO: 9. The whole vector sequence is shown. The cDNA of the DZS18 can be inserted in frame with the C-terminus end of the malE gene creating a malE-DZS18 fusion protein.

SEQ ID NO: 9:
CGACACCATCGAATGGTGCAAAACCTTTCGCGGTATGGCATGATAGCG
CCCGGAAGAGAGTCAATTCAGGGTGGTGAATGTGAAACCAGTAACGTT
ATACGATGTCGCAGAGTATGCCGGTGTCTCTTATCAGACCGTTTCCCG
CGTGGTGAACCAGGCCAGCCACGTTTCTGCGAAAACGCGGGAAAAAGT
GGAAGCGGCGATGGCGGAGCTGAATTACATTCCCAACCGCGTGGCACA
ACAACTGGCGGGCAAACAGTCGTTGCTGATTGGCGTTGCCACCTCCAG
TCTGGCCCTGCACGCGCCGTCGCAAATTGTCGCGGCGATTAAATCTCG
CGCCGATCAACTGGGTGCCAGCGTGGTGGTGTCGATGGTAGAACGAAG
CGGCGTCGAAGCCTGTAAAGCGGCGGTGCACAATCTTCTCGCGCAACG
CGTCAGTGGGCTGATCATTAACTATCCGCTGGATGACCAGGATGCCAT
TGCTGTGGAAGCTGCCTGCACTAATGTTCCGGCGTTATTTCTTGATGT
CTCTGACCAGACACCCATCAACAGTATTATTTTCTCCCATGAAGACGG
TACGCGACTGGGCGTGGAGCATCTGGTCGCATTGGGTCACCAGCAAAT
CGCGCTGTTAGCGGGCCCATTAAGTTCTGTCTCGGCGCGTCTGCGTCT
GGCTGGCTGGCATAAATATCTCACTCGCAATCAAATTCAGCCGATAGC
GGAACGGGAAGGCGACTGGAGTGCCATGTCCGGTTTTCAACAAACCAT
GCAAATGCTGAATGAGGGCATCGTTCCCACTGCGATGCTGGTTGCCAA
CGATCAGATGGCGCTGGGCGCAATGCGCGCCATTACCGAGTCCGGGCT
GCGCGTTGGTGCGGATATTTCGGTAGTGGGATACGACGATACCGAAGA
CAGCTCATGTTATATCCCGCCGTTAACCACCATCAAACAGGATTTTCG
CCTGCTGGGGCAAACCAGCGTGGACCGCTTGCTGCAACTCTCTCAGGG
CCAGGCGGTGAAGGGCAATCAGCTGTTGCCCGTCTCACTGGTGAAAAG
AAAAACCACCCTGGCGCCCAATACACAAACCGCCTCTCCCCGCGCGTT
GGCCGATTCATTAATGCAGCTGGCACGACAGGTTTCCCGACTGGAAAG
CGGGCAGTGAGCGCAACGCAATTAATGTAAGTTAGCTCACTCATTAGG
CACAATTCTCATGTTTGACAGCTTATCATCGACTGCACGGTGCACCAA
TGCTTCTGGCGTCAGGCAGCCATCGGAAGCTGTGGTATGGCTGTGCAG
GTCGTAAATCACTGCATAATTCGTGTCGCTCAAGGCGCACTCCCGTTC
TGGATAATGTTTTTTGCGCCGACATCATAACGGTTCTGGCAAATATTC
TGAAATGAGCTGTTGACAATTAATCATCGGCTCGTATAATGTGTGGAA
TTGTGAGCGGATAACAATTTCACACAGGAAACAGCCAGTCCGTTTAGG
TGTTTTCACGAGCAATTGACCAACAAGGACCATAGATTATGAAAATCC
ACCATCACCACCACCACGAAGAAGGTAAACTGGTAATCTGGATTAACG
GCGATAAAGGCTATAACGGTCTCGCTGAAGTCGGTAAGAAATTCGAGA
AAGATACCGGAATTAAAGTCACCGTTGAGCATCCGGATAAACTGGAAG
AGAAATTCCCACAGGTTGCGGCAACTGGCGATGGCCCTGACATTATCT
TCTGGGCACACGACCGCTTTGGTGGCTACGCTCAATCTGGCCTGTTGG
CTGAAATCACCCCGGACAAAGCGTTCCAGGACAAGCTGTATCCGTTTA
CCTGGGATGCCGTACGTTACAACGGCAAGCTGATTGCTTACCCGATCG
CTGTTGAAGCGTTATCGCTGATTTATAACAAAGATCTGCTGCCGAACC
CGCCAAAAACCTGGGAAGAGATCCCGGCGCTGGATAAAGAACTGAAAG
CGAAAGGTAAGAGCGCGCTGATGTTCAACCTGCAAGAACCGTACTTCA
CCTGGCCGCTGATTGCTGCTGACGGGGGTTATGCGTTCAAGTATGAAA
ACGGCAAGTACGACATTAAAGACGTGGGCGTGGATAACGCTGGCGCGA
AAGCGGGTCTGACCTTCCTGGTTGACCTGATTAAAAACAAACACATGA
ATGCAGACACCGATTACTCCATCGCAGAAGCAGCCTTTAATAAAGGCG
AAACAGCGATGACCATCAACGGCCCGTGGGCATGGTCCAACATCGACA
CCAGCAAAGTGAATTATGGTGTAACGGTACTGCCGACCTTCAAGGGTC
AACCATCCAAACCGTTCGTTGGCGTGCTGAGCGCAGGTATTAACGCCG
CCAGTCCGAACAAAGAGCTGGCAAAAGAGTTCCTCGAAAACTATCTGC
TGACTGATGAAGGTCTGGAAGCGGTTAATAAAGACAAACCGCTGGGTG
CCGTAGCGCTGAAGTCTTACGAGGAAGAGTTGGTGAAAGATCCGCGTA
TTGCCGCCACTATGGAAAACGCCCAGAAAGGTGAAATCATGCCGAACA
TCCCGCAGATGTCCGCTTTCTGGTATGCCGTGCGTACTGCGGTGATCA
ACGCCGCCAGCGGTCGTCAGACTGTCGATGAAGCCCTGAAAGACGCGC
AGACTAATTCGAGCTCGAACAACAACAACAATAACAATAACAACAACC
TCGGGGAGAACCTGTACTTCCAGATGCTGATGGGCGGCCGCGATATCG
TCGACGGATCCGAATTCCCTGCAGGTAATTAAATAAGCTTCAAATAAA
ACGAAAGGCTCAGTCGAAAGACTGGGCCTTTCGTTTTATCTGTTGTTT
GTCGGTGAACGCTCTCCTGAGTAGGACAAATCCGCCGGGAGCGGATTT
GAACGTTGCGAAGCAACGGCCCGGAGGGTGGGGGCAGGACGCCCGCCA
TAAACTGCCAGGCATCAAATTAAGCAGAAGGCCATCCTGACGGATGGC
CTTTTTGCGTTTCTACAAACTCTTTCGGTCCGTTGTTTATTTTTCTAA
ATACATTCAAATATGTATCCGCTCATGAGACAATAACCCTGATAAATG
CTTCAATAATATTGAAAAAGGAAGAGTATGAGTATTCAACATTTCCGT
GTCGCCCTTATTCCCTTTTTTGCGGCATTTTGCCTTCCTGTTTTTGCT
CACCCAGAAACGCTGGTGAAAGTAAAAGATGCTGAAGATCAGTTGGGT
GCACGAGTGGGTTACATCGAACTGGATCTCAACAGCGGTAAGATCCTT
GAGAGTTTTCGCCCCGAAGAACGTTTCCCAATGATGAGCACTTTTAAA
GTTCTGCTATGTGGCGCGGTATTATCCCGTGTTGACGCCGGGCAAGAG
CAACTCGGTCGCCGCATACACTATTCTCAGAATGACTTGGTTGAGTAC
TCACCAGTCACAGAAAAGCATCTTACGGATGGCATGACAGTAAGAGAA
TTATGCAGTGCTGCCATAACCATGAGTGATAACACTGCGGCCAACTTA
CTTCTGACAACGATCGGAGGACCGAAGGAGCTAACCGCTTTTTTGCAC
AACATGGGGGATCATGTAACTCGCCTTGATCGTTGGGAACCGGAGCTG
AATGAAGCCATACCAAACGACGAGCGTGACACCACGATGCCTGTAGCA
ATGGCAACAACGTTGCGCAAACTATTAACTGGCGAACTACTTACTCTA
GCTTCCCGGCAACAATTAATAGACTGGATGGAGGCGGATAAAGTTGCA
GGACCACTTCTGCGCTCGGCCCTTCCGGCTGGCTGGTTTATTGCTGAT
AAATCTGGAGCCGGTGAGCGTGGGTCTCGCGGTATCATTGCAGCACTG
GGGCCAGATGGTAAGCCCTCCCGTATCGTAGTTATCTACACGACGGGG
AGTCAGGCAACTATGGATGAACGAAATAGACAGATCGCTGAGATAGGT
GCCTCACTGATTAAGCATTGGTAACTGTCAGACCAAGTTTACTCATAT
ATACTTTAGATTGATTTCCTTAGGACTGAGCGTCAACCCCGTAGAAAA
GATCAAAGGATCTTCTTGAGATCCTTTTTTTCTGCGCGTAATCTGCTG
CTTGCAAACAAAAAAACCACCGCTACCAGCGGTGGTTTGTTTGCCGGA
TCAAGAGCTACCAACTCTTTTTCCGAAGGTAACTGGCTTCAGCAGAGC
GCAGATACCAAATACTGTCCTTCTAGTGTAGCCGTAGTTAGGCCACCA
CTTCAAGAACTCTGTAGCACCGCCTACATACCTCGCTCTGCTAATCCT
GTTACCAGTGGGTGCTGCCAGTGGCGATAAGTCGTGTCTTACCGGGTT
GGACTCAAGACGATAGTTACCGGATAAGGCGCAGCGGTCGGGCTGAAC
GGGGGGTTCGTGCACACAGCCCAGCTTGGAGCGAACGACCTACACCGA
ACTGAGATACCTACAGCGTGAGCTATGAGAAAGCGCCACGCTTCCCGA
AGGGAGAAAGGCGGACAGGTATCCGGTAAGCGGCAGGGTCGGAACAGG
AGAGCGCACGAGGGAGCTTCCAGGGGGAAACGCCTGGTATCTTTATAG
TCCTGTCGGGTTTCGCCACCTCTGACTTGAGCGTCGATTTTTGTGATG
CTCGTCAGGGGGGCGGAGCCTATGGAAAAACGCCAGCAACGCGGCCTT
TTTACGGTTCCTGGCCTTTTGCTGGCCTTTTGCTCACATGTTCTTTCC
TGCGTTATCCCCTGATTCTGTGGATAACCGTATTACCGCCTTTGAGTG
AGCTGATACCGCTCGCCGCAGCCGAACGACCGAGCGCAGCGAGTCAGT
GAGCGAGGAAGCGGAAGAGCGCCTGATGCGGTATTTTCTCCTTACGCA
TCTGTGCGGTATTTCACACCGCATATAAGGTGCACTGTGACTGGGTCA
TGGCTGCGCCCCGACACCCGCCAACACCCGCTGACGCGCCCTGACGGG
CTTGTCTGCTCCCGGCATCCGCTTACAGACAAGCTGTGACCGTCTCCG
GGAGCTGCATGTGTCAGAGGTTTTCACCGTCATCACCGAAACGCGCGA
GGCAGCTGCGGTAAAGCTCATCAGCGTGGTCGTGCAGCGATTCACAGA
TGTCTGCCTGTTCATCCGCGTCCAGCTCGTTGAGTTTCTCCAGAAGCG
TTAATGTCTGGCTTCTGATAAAGCGGGCCATGTTAAGGGCGGTTTTTT
CCTGTTTGGTCACTGATGCCTCCGTGTAAGCTCAGGCCAGGACCCAAC
GCTGCCCGAAATTC

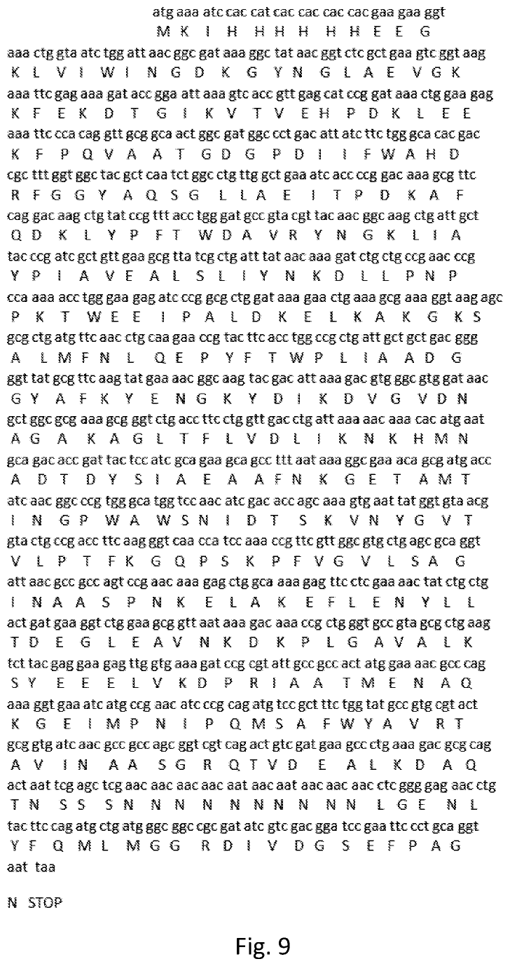

The sequence of the MalE gene in the pMAL-c6T vector (e.g., MBP region) is provided in FIG. 9, with the nucleic acid sequence being SEQ NO: 10, and the amino acid sequence being SEQ ID NO: 11. This SEQ ID NO: 11 can be for the N-terminus 6His-Maltose-Binding Protein fusion protein (6-His-MBP polypeptide)

SEQ ID NO: 10:
atg aaa atc cac cat cac cac cac cac gaa gaa ggt
aaa ctg gta atc tgg att aac ggc gat aaa ggc tat
aac ggt ctc gct gaa gtc ggt aag aaa ttc gag aaa
gat acc gga att aaa gtc acc gtt gag cat ccg gat
aaa ctg gaa gag aaa ttc cca cag gut gcg gca act
ggc gat ggc cct gac att atc ttc tgg gca cac gac
cgc ttt ggt ggc tac gct caa tct ggc ctg ttg gct
gaa atc acc ccg gac aaa gcg ttc cag gac aag ctg
tat ccg ttt acc tgg gat gcc gta cgt tac aac ggc
aag ctg att gct tac ccg atc gct gtt gaa gcg tta
tcg ctg att tat aac aaa gat ctg ctg ccg aac ccg
cca aaa acc tgg gaa gag atc ccg gcg ctg gat aaa
gaa ctg aaa gcg aaa ggt aag agc gcg ctg atg ttc
aac ctg caa gaa ccg tac ttc acc tgg ccg ctg att
gct gct gac ggg ggt tat gcg ttc aag tat gaa aac
ggc aag tac gac att aaa gac gtg ggc gtg gat aac
gct ggc gcg aaa gcg ggt ctg acc ttc ctg gtt gac
ctg att aaa aac aaa cac atg aat gca gac acc gat
tac tcc atc gca gaa gca gcc ttt aat aaa ggc gaa
aca gcg atg acc atc aac ggc ccg tgg gca tgg tcc
aac atc gac acc agc aaa gtg aat tat ggt gta acg
gta ctg ccg acc ttc aag ggt caa cca tcc aaa ccg
ttc gtt ggc gtg ctg agc gca ggt att aac gcc gcc
agt ccg aac aaa gag ctg gca aaa gag ttc ctc gaa
aac tat ctg ctg act gat gaa ggt ctg gaa gcg gtt
aat aaa gac aaa ccg ctg ggt gcc gta gcg ctg aag
tct tac gag gaa gag ttg gtg aaa gat ccg cgt att
gcc gcc act atg gaa aac gcc cag aaa ggt gaa atc
atg ccg aac atc ccg cag atg tcc gct ttc tgg tat
gcc gtg cgt act gcg gtg atc aac gcc gcc agc ggt
cgt cag act gtc gat gaa gcc ctg aaa gac gcg cag
act aat tcg agc tcg aac aac aac aac aat aac aat
aac aac aac ctc ggg gag aac ctg tac ttc cag atg
ctg atg ggc ggc cgc gat atc gtc gac gga tcc gaa
ttc cct gca ggt aat taa
SEQ ID NO: 11:
M K I H H H H H H E E G K L V I W I N G D K G Y
N G L A E V G K K F E K D T G I K V T V E H P D
K L E E K F P Q V A A T G D G P D I I F W A H D
R F G G Y A Q S G L L A E I T P D K A F Q D K L
Y P F T W D A V R Y N G K L I A Y P I A V E A L
S L I Y N K D L L P N P P K T W E E I P A L D K
E L K A K G K S A L M F N L Q E P Y F T W P L I
A A D G G Y A F K Y E N G K Y D I K D V G V D N
A G A K A G L T F L V D L I K N K H M N A D T D
Y S I A E A A F N K G E T A M T I N G P W A W S
N I D T S K V N Y G V T V L P T F K G Q P S K P
F V G V L S A G I N A A S P N K E L A K E F L E
N Y L L T D E G L E A V N K D K P L G A V A L K
S Y E E E L V K D P R I A A T M E N A Q K G E I
M P N I P Q M S A F W Y A V R T A V I N A A S G
R Q T V D E A L K D A Q T N S S S N N N N N N N
N N N L G E N L Y F Q M L M G G R D I V D G S E
F P A G N

The final product Met-rich fusion protein is SEQ ID NO: 12. This fusion protein can be cleaved to obtain SEQ ID NO: 8.

SEQ ID NO: 12:
M K I H H H H H H E E G K L V I W I N G D K G Y
N G L A E V G K K F E K D T G I K V T V E H P D
K L E E K F P Q V A A T G D G P D I I F W A H D
R F G G Y A Q S G L L A E I T P D K A F Q D K L
Y P F T W D A V R Y N G K L I A Y P I A V E A L
S L I Y N K D L L P N P P K T W E E I P A L D K
E L K A K G K S A L M E N L Q E P Y F T W P L I
A A D G G Y A F K Y E N G K Y D I K D V G V D N
A G A K A G L T F L V D L I K N K H M N A D T D
Y S I A E A A F N K G E T A M T I N G P W A W S
N I D T S K V N Y G V T V L P T F K G Q P S K P
F V G V L S A G I N A A S P N K E L A K E F L E
N Y L L T D E G L E A V N K D K P L G A V A L K
S Y E E E L V K D P R I A A T M E N A Q K G E I
M P N I P Q M S A F W Y A V R T A V I N A A S G
R Q T V D E A L K D A Q T N S S S N N N N N N N
N N N L G E N L Y F Q M L M G G R D I Met A A K
M F A L F A L L A L C A T A T S A T H I Q A L V
T T T D A I G Y H E P W M Q Y C M K Q Q G V A N
L L A W P T L M L Q Q L L A S P L Q Q C Q M P M
M M P G M M P P M T M M P M P S M M P S M V P T
M M S P M T M A S M M P P M M M P S M I S P M T
M P S M M P S M I M P T M M S P M I M P S M M P
P M M M P S M V S P M M M P N M M T V P Q C Y S
G S I S H I I Q Q Q Q L P F M F S P T A M A I P
P M F L Q Q P F V G A A F

Underlined sequence: TEV cleaving site. Met: starting of DZS18.

In some embodiments, a plasmid is provided having a sequence that encodes for a polypeptide of SEQ ID NOs: 1 or 12 so as to have at least 80%, 90%, 95%, or 99% identity therewith. In some aspects, the plasmid may encode for a 6His-MBP polypeptide tag by having a nucleic acid sequence of is SEQ ID NO: 10 so as to have at least 80%, 90%, 95%, or 99% identity therewith, or be complementary thereto. The plasmid may include a sequence that encodes for a 6His-MBP polypeptide tag having the polypeptide SEQ ID NO: 11 so as to have at least 80%, 90%, 95%, or 99% identity therewith. The plasmid may include a sequence that encodes for a Met-rich polypeptide as is SEQ ID NO: 2, 3, 8, or 13 so as to have at least 80%, 90%, 95%, or 99% identity therewith. In some aspects, a cell, such as a bacteria cell, can include the plasmid, and can be used in a method of encoding for the corresponding hybrid polypeptide. Accordingly, a cell culture, such as bacteria, can be grown that includes cells with the plasmid, and the hybrid polypeptide can be purified therefrom.

In some embodiments, a hybrid polypeptide having a 6His-MBP polypeptide tag and Met-rich polypeptide can have the sequence of SEQ ID NO: 1 or 12, or at least 80%, 90%, 95%, or 99% identity therewith. The hybrid polypeptide may include a sequence of a 6His-MBP polypeptide tag having the polypeptide SEQ ID NO:11 so as to have at least 80%, 90%, 95%, or 99% identity therewith. The hybrid polypeptide may include a sequence of a Met-rich polypeptide having the polypeptide SEQ ID NO: 2, 3, 8, or 13 so as to have at least 80%, 90%, 95%, or 99% identity therewith. In some aspects, a cell, such as a bacteria cell, can include the hybrid polypeptide by production from a plasmid therein. The cell can release the hybrid polypeptide, or the hybrid polypeptide can be extracted from the cell.

In some embodiments, a primer is provided that has a sequence that hybridizes with the plasmid that encodes for the polypeptide of SEQ ID NOs: 1 or 12. In some embodiments, a probe is provided that has a sequence that hybridizes with the plasmid that encodes for the polypeptide of SEQ ID NOs: 1 or 12. The probe can have a label, wherein the label is configured to be detected. A primer or probe having the sequence of SEQ ID NO: 5, or complement thereof, or at least 80%, 90%, 95%, or 99% identity therewith. A primer or probe having the sequence of SEQ ID NO: 6, or complement thereof, or at least 80%, 90%, 95%, or 99% identity therewith.

In some embodiments, a nucleic acid that encodes for the 6His-MBP polypeptide is SEQ ID NO: 10. The protein sequence of the 6His-MBP polypeptide is SEQ ID NO:11. The Met-rich polypeptide is SEQ ID NO: 2, 3, 8, or 13.

In some embodiments, the nucleic acids or polypeptides may be isolated from a cell or biological sample.

### Definitions

Unless defined otherwise, all technical and scientific terms used herein have the same meaning as commonly understood by one of ordinary skill in the art to which the present disclosure belongs.

The term “isolated” means that the material is removed from its original environment (e.g., the natural environment if it is naturally occurring). For example, a naturally occurring antibody present in a living animal is not isolated, but the same antibody, separated from some or all of the coexisting materials in the natural system, is isolated.

As used herein, the term “antibody” refers to intact molecules as well as fragments thereof, such as Fab, F(ab′) 2, and Fv, which are capable of binding the epitopic determinant. Methods of making these fragments are known in the art. The term “antibody” includes monoclonal antibodies, polyclonal antibodies, chimeric, or humanized antibodies. The antibodies of the invention can be of any type (e.g., IgG, IgE, IgM, IgD, IgA, and IgY), class (e.g., IgG1, IgG2, IgG3, IgG4, IgA1, and IgA2), or subclass of immunoglobulin molecule.

The antibodies of the present invention may be generated by any suitable method known in the art. Polyclonal antibodies to the antigen of interest can be produced by various procedures well known in the art. For example, the oxidized zein proteins containing numerous MetO residues can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught. The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. In a non-limiting example, mice or rabbits can be immunized with the MetO-containing zein proteins of the present invention. Once an immune response is detected, e.g., antibodies specific for the antigen are detected in the mouse serum, the mouse spleen is harvested and splenocytes isolated. The splenocytes are then fused by well-known techniques to any suitable myeloma cells, for example, cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention.

If desired, polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies.

As used herein, the term “specific for” when used in reference to the interaction of an antibody protein or peptide means that the interaction is dependent upon the presence of a particular structure (i.e., the antigenic determinant or epitope) on the protein; in other words the antibody is recognizing and binding to a specific protein structure rather than to proteins in general (i.e. non-specific or background binding).

The term “nucleic acid(s)” as used in this invention refers to, for example, DNA or RNA, or polynucleotides derived therefrom which are active as DNA or RNA, and preferably they are DNA or RNA.

The term “isolated nucleic acid(s)” as used in the present specification refers to a nucleic acid or a polynucleotide containing substantially no cellular substances or culture medium, if prepared by recombinant DNA techniques, or containing substantially no precursor chemical substances or other chemical substances, if prepared by chemical synthesis.

The terms “peptide,” “polypeptide” and “protein” are used interchangeably herein.

As used herein, an “isolated polypeptide” (e.g., isolated His-MBP-Met-rich hybrid polypeptide) is intended to mean a polypeptide that has been completely or partially removed from its native environment. For example, polypeptides that have been removed or purified from cells are considered isolated. In addition, recombinantly produced polypeptides molecules contained in host cells are considered isolated for the purposes of the present invention. Moreover, a peptide that is found in a cell, tissue or matrix in which it is not normally expressed or found is also considered as “isolated” for the purposes of the present invention. Similarly, polypeptides that have been synthesized are considered to be isolated polypeptides. “Purified,” on the other hand is well understood in the art and generally means that the peptides are substantially free of cellular material, cellular components, chemical precursors or other chemicals beyond, perhaps, buffer or solvent. “Substantially free” is not intended to mean that other components beyond the novel peptides are undetectable. The peptides of the present invention may be isolated or purified.

A polypeptide having an amino acid sequence at least, for example, about 95% “identical” to a reference an amino acid sequence is understood to mean that the amino acid sequence of the polypeptide is identical to the reference sequence except that the amino acid sequence may include up to about five modifications per each 100 amino acids of the reference amino acid sequence, or relative amount for shorter sequences. In other words, to obtain a peptide having an amino acid sequence at least about 95% identical to a reference amino acid sequence, up to about 5% of the amino acid residues of the reference sequence may be deleted or substituted with another amino acid or a number of amino acids up to about 5% of the total amino acids in the reference sequence may be inserted into the reference sequence. These modifications of the reference sequence may occur at the N-terminus or C-terminus positions of the reference amino acid sequence or anywhere between those terminal positions, interspersed either individually among amino acids in the reference sequence or in one or more contiguous groups within the reference sequence.

As used herein, “identity” is a measure of the identity of nucleotide sequences or amino acid sequences compared to a reference nucleotide or amino acid sequence. In general, the sequences are aligned so that the highest order match is obtained. “Identity” per se has an art-recognized meaning and can be calculated using well known techniques. While there are several methods to measure identity between two polynucleotide or polypeptide sequences, the term “identity” is well known to skilled artisans.

As used herein, the term “subject” includes humans capable of suffering from, suffering from, or having symptoms of a disease state associated with MetO proteins, such as a neurodegenerative disease (AD), as well as any human seeking a prophylactic to such MetO protein mediated disease states.

As used herein, the term “treat” or “treated” or “treating” or “treatment” refer to any type of action that imparts a modulating effect, which, for example, can be a beneficial effect, to a subject afflicted with a disorder, disease, or condition (e.g., neurodegenerative disease or symptoms), including improvement in the condition of the subject (e.g., in one or more symptoms), delay or reduction in the progression of the condition, and/or change in clinical parameters, disease or illness, etc., as would be well known in the art. The term “treatment” includes therapeutic and/or prophylactic treatment of Alzheimer's disease, the diminishment or alleviation of at least one symptom associated with Alzheimer's disease (e.g., due to MetO proteins or amyloids), and the eradication of one or more symptoms of Alzheimer's disease.

As used herein, “prevent” or “prevented” or “preventing” or “prevention” refer to prevention or delay of the onset of a disorder, disease, or condition (e.g., Alzheimer's disease) and/or a decrease in the symptoms of a disease in a subject relative to the symptoms of neurodegenerative disease that would develop in the absence of the methods of the invention. The prevention can be complete, for example, the total absence of disease in a subject. The prevention can also be partial, such that the neurodegenerative disease in a subject has reduced symptoms from that which would have occurred without the present invention. The terms “prevention”, “prophylactic treatment”, and “prophylaxis” may be used interchangeably and are intended to refer to prevention.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

### REFERENCES

1. Selkoe, D. J.; Hardy, J. The amyloid hypothesis of Alzheimer's disease at 25 years. EMBO Mol. Med. 2016, 8, 595-608.
2. Sultana, R.; Butterfield, D. A. Role of oxidative stress in the progression of Alzheimer's disease. J. Alzheimer's Dis. 2010, 19, 341-353.
3. Halliwell, B. Role of free radicals in the neurodegenerative diseases: Therapeutic implications for antioxidant treatment. Drugs Aging 2001, 18, 685-716.
4. Terman, A.; Brunk, U. T. Oxidative stress, accumulation of biological ‘garbage’, and aging. Antioxid. Redox Signal. 2006, 8, 197-204.
5. Nunomura, A.; Perry, G.; Pappolla, M. A.; Wade, R.; Hirai, K.; Chiba, S.; Smith, M. A. RNA oxidation is a prominent feature of vulnerable neurons in Alzheimer's disease. J. Neurosci. 1999, 19, 1959-1964.
6. Selkoe, D. J. Alzheimer's disease: Genes, proteins, and therapy. Physiol. Rev. 2001, 81, 741-766.
7. Hardy, J. A.; Higgins, G. A. Alzheimer's disease: The amyloid cascade hypothesis. Science 1992, 256, 184-185.
8. Roychaudhuri, R.; Yang, M.; Hoshi, M. M.; Taplow, D. B. Amyloid beta-protein assembly and Alzheimer disease. J. Biol. Chem. 2009, 284, 4749-4753.
9. Nunomura, A.; Perry, G.; Aliev, G.; Hirai, K.; Takeda, A.; Balraj, E. K.; Jones, P. K.; Ghanbari, H.; Wataya, T.; Shimohama, S.; et al., Oxidative damage is the earliest event in Alzheimer disease. J. Neuropathol. Exp. Neurol. 2001, 60, 759-767.
10. Hirai, K.; Aliev, G.; Nunomura, A.; Fujioka, H.; Russell, R. L.; Atwood, C. S.; Johnson, A. B.; Kress, Y.; Vinters, H. V.; Tabaton, M.; et al. Mitochondrial abnormalities in Alzheimer's disease. J. Neurosci. 2001, 21, 3017-3023.
11. Dumont, M.; Lin, M. T.; Beal, M. F. Mitochondria and antioxidant targeted therapeutic strategies for Alzheimer's disease. J. Alzheimer's Dis. 2010, 20 (Suppl. 2), S633-S643.
12. Bitan, G.; Tarus, B.; Vollers, S. S.; Lashuel, H. A.; Condron, M. M.; Straub, J. E.; Teplow, D. B. A molecular switch in amyloid assembly: Met35 and amyloid beta-protein oligomerization. J. Am. Chem. Soc. 2003, 25, 15359-15365;
13. Näslund, J.; Schierhorn, A.; Hellman, U.; Lannfelt, L.; Roses, A. D.; Tjernberg, L. O.; Silberring, L.; Gandy, S. E.; Winblad, B.; Greengard, P.; et al. Relative abundance of Alzheimer A beta amyloid peptide variants in Alzheimer disease and normal aging. Proc. Natl. Acad. Sci. USA 1994, 91, 8378-8382.
14. Kuo, Y. M.; Kokjohn, T. A.; Beach, T. G.; Sue, L. I.; Brune, D.; Lopez, J. C.; Kalback, W. M.; Abramowski, D.; Sturchler-Pierrat, C.; Staufenbiel, M.; et al. Comparative analysis of amyloid-beta chemical structure and amyloid plaque morphology of transgenic mouse and Alzheimer's disease brains. J. Biol. Chem. 2001, 276, 12991-12998.
15. Dong, J.; Atwood, C. S.; Anderson, V. E.; Siedlak, S. L.; Smith, M. A.; Perry, G.; Carey, P. R. Metal binding and oxidation of amyloid-beta within isolated senile plaque cores: Raman microscopic evidence. Biochemistry 2003, 42, 2768-2773; 17-Boutte, A. M.; Woltjer, R. L.; Zimmerman, L. J.; Stamer, S. L.; Montine, K. S.; Manno, M. V.; Cimino, P. J.; Liebler, D. C.; Montine, T. J. Selectively increased oxidative modifications mapped to detergent-insoluble forms of Aβ and β-III tubulin in Alzheimer's disease. FASEB J. 2006, 20, 1473-1483.
16. Butterfield, D. A.; Boyd-Kimball, D. The critical role of methionine 35 in Alzheimer's amyloid beta-peptide (1-42)-induced oxidative stress and neurotoxicity. Biochim. Biophys. Acta 2005, 1703, 149-156.
17. Triguero, L.; Singh, R.; Prabhakar, R. Comparative molecular dynamics studies of wild-type and oxidized forms of full-length Alzheimer amyloid beta-peptides Aβ(1-40) and Aβ(1-42). J. Phys. Chem. B 2008, 112, 7123-7131.
18. Moskovitz, J.; Maiti, P.; Lopes, D. H.; Oien, D. B.; Attar, A.; Liu, T.; Mittal, S.; Hayes, J.; Bitan, G. Induction of methionine-sulfoxide reductases protects neurons from amyloid β-protein insults in vitro and in vivo. Biochemistry 2011, 50, 10687-10697.
19. Stadtman, E. R.; Moskovitz, J.; Berlett, B. S.; Levine, R. L. Cyclic oxidation and reduction of protein methionine residues is an important antioxidant mechanism. Mol. Cell. Biochem. 2002, 234, 3-9.
20. Oien, D. B.; Canello, T.; Gabizon, R.; Gasset, M.; Lundquist, B. L.; Burns, J. M.; Moskovitz, J. Detection of oxidized methionine in selected proteins, cellular extracts and blood serums by novel anti-methionine sulfoxide antibodies. Arch. Biochem. Biophys. 2009, 485, 35-40.
21. Hu, J.; Qin, H.; Gao, F. P.; Cross, T. A. A systematic assessment of mature MBP in membrane protein production: Overexpression, membrane targeting and purification. Protein. Expr. Purif. 2011, 80, 34-40.
22. Webster, S. J.; Bachstetter, A. D.; Nelson, P. T.; Schmitt, F. A.; Van Eldik, L. J. Using mice to model Alzheimer's dementia: An overview of the clinical disease and the preclinical behavioral changes in 10 mouse models. Front Genet. 2014, 5, 88. doi.org/10.3389/fgene.2014.00088.
23. Gallagher, J. J.; Minogue, A. M.; Lynch, M. A. Impaired performance of female APP/PS1 mice in the Morris water maze is coupled with increased Aβ accumulation and microglial activation. Neurodegener. Dis. 2013, 11, 33-41.
24. Pal, R.; Oien, D. B.; Ersen, F. Y.; Moskovitz, J. Elevated levels of brain-pathologies associated with neurodegenerative diseases in the methionine sulfoxide reductase A knockout mouse. Exp. Brain Res. 2007, 180, 765-774.
25. Schmidlin, C. J.; Dodson, M. B.; Madhavan, L.; Zhang, D. D. Redox regulation by NRF2 in aging and disease. Free Radic. Biol. Med. 2019, 134, 702-707.
26. Dieckman L, Gu M, Stols L, Donnelly MI, Collart FR. High throughput methods for gene cloning and expression. Protein Expr Purif. 2002; 25:1-7 . . .
27. Qin H, Hu J, Hua Y, Challa SV, Cross TA, Gao FP. Construction of a series of vectors for high throughput cloning and expression screening of membrane proteins from Mycobacterium tuberculosis. BMC Biotechnol. 2008; 8:51
28. J. Hu, H. Qin, F. P. Gao, T. A. Cross, A systematic assessment of mature MBP in membrane protein production: overexpression, membrane targeting and purification. Protein Expr. Purif. 80 (1), 34-40 (2011

1. A plasmid having a sequence that encodes for a hybrid polypeptide, the hybrid polypeptide comprising:
a histidine tag region;
a solubility promoter region adjacent to the histidine tag region;
a methionine-rich region, wherein the solubility promoter region is between the histidine tag and the methionine-rich region; and
at least one cleavage site between the solubility promoter region and the methionine-rich region, such that cleavage produces a methionine-rich polypeptide.

2. The plasmid of claim 1, wherein the encoded hybrid polypeptide has a polypeptide sequence of SEQ ID NO: 1 or SEQ ID NO: 12, at least about 80% identity therewith.

3. The plasmid of claim 1, wherein at least one of:
the histidine tag is N-terminal and has 6 consecutive histidine residues and the solubility promoter region is MBP in a 6-His-MBP polypeptide having a SEQ ID NO: 11, or at least about 80% identity therewith; or
the methionine-rich region is SEQ ID NO: 2, 3, 8, or 13, or at least about 80% identity therewith.

4. A cell comprising the plasmid of claim 1.

5. A hybrid polypeptide comprising:
a histidine tag region;
a solubility promoter region adjacent to the histidine tag region;
a methionine-rich region, wherein the solubility promoter region is between the histidine tag and the methionine-rich region; and
at least one cleavage site between the solubility promoter region and the methionine-rich region, such that cleavage produces a methionine-rich polypeptide.

6. The hybrid polypeptide of claim 5, wherein at least one of:
the histidine tag has at least about 6 consecutive histidine residues;
the solubility promoter region is selected from MBP, GST, SUMO, or GB1;
the methionine-rich region has at least 20% amino acids being methionine; or
at least one cleavage site is a protease cleavage site.

7. The hybrid polypeptide of claim 5, wherein at least one of:
the histidine tag is N-terminal and has about 6 consecutive histidine residues;
the solubility promoter region is MBP;
the methionine-rich region has at least about 24% amino acids being methionine; or
at least one cleavage site is a protease cleavage site.

8. The hybrid polypeptide of claim 5, comprising a polypeptide sequence of SEQ ID NO: 1 or SEQ ID NO: 12, or at least about 80% identity therewith.

9. The hybrid polypeptide of claim 5, wherein at least one of:
the histidine tag is N-terminal and has 6 consecutive histidine residues and the solubility promoter region is MBP in a 6-His-MBP polypeptide having a SEQ ID NO: 11, or at least about 80% identity therewith; or
the methionine-rich region is SEQ ID NO: 2, 3, 8 or 13, or at least about 80% identity therewith.

10. An anti-methionine sulfoxide antibody that binds with a methionine sulfoxide-rich polypeptide that includes the sequence of SEQ ID NO: 2, 3, 8, or 13, or at least about 80% identity therewith.

11. An immunization composition comprising:
a methionine sulfoxide-rich polypeptide antigen derived from a methionine sulfoxide-rich polypeptide that includes the sequence of SEQ ID NO: 2, 3, 8, or 13, or at least about 80% identity therewith, wherein the methionine sulfide-rich polypeptide includes a majority of methionine residues being oxidated; and
an adjuvant.

12. A method of producing a methionine-rich polypeptide, comprising:
providing the plasmid of claim 1;
producing the hybrid polypeptide from the plasmid; and
cleaving the methionine-rich region from the hybrid polypeptide to produce the methionine-rich polypeptide.

13. A method of producing a methionine-rich polypeptide, comprising:
providing the hybrid polypeptide of claim 7; and
cleaving the methionine-rich region from the hybrid polypeptide to produce the methionine-rich polypeptide.

14. A method of producing a methionine sulfoxide-rich polypeptide, comprising:
providing the plasmid of claim 1;
producing the hybrid polypeptide from the plasmid;
cleaving the methionine-rich region from the hybrid polypeptide to produce the methionine-rich polypeptide; and
oxidizing a plurality of methionine residues in the methionine-rich polypeptide to form the methionine sulfoxide-rich polypeptide.

15. A method of producing a methionine-rich polypeptide, comprising:
providing the hybrid polypeptide of claim 7;
cleaving the methionine-rich region from the hybrid polypeptide to produce the methionine-rich polypeptide; and
oxidizing a plurality of methionine residues in the methionine-rich polypeptide to form the methionine sulfoxide-rich polypeptide.

16. A method of immunizing a subject against methionine sulfoxide-containing proteins, comprising:
obtaining the methionine sulfoxide-rich polypeptide according to the method of claim 15; and
immunizing the subject against the methionine sulfoxide-containing proteins by administration of the methionine sulfoxide-rich polypeptide,
wherein the immunizing of the subject is sufficient to at least one of:
inhibit onset or progression of a neurodegenerative disease, which is optionally Alzheimer's disease;
inhibit onset or progression of a learning disorder;
inhibit onset or progression of memory loss;
produce anti-methionine sulfoxide antibodies
improving cognitive function;
of increasing clearance of methionine sulfoxide-containing proteins;

17. The method of claim 16, wherein the methionine sulfoxide-containing proteins includes an amyloid or Apo-J having an oxidized methionine residue.

18. The method of claim 16, wherein production of new Apo-J is induced due to clearance of Apo-J having an oxidized methionine residue.


SEQUENCE LISTING (13 sequences)

SEQ ID NO: 1 - protein, 574 aa, synthetic construct
MHHHHHHSTSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGTENLYFQSNAHIQALVTTTDAIGYHEPWMQYCMKQQGVANLLAWPTLMLQQLLASPLQQCQMPMMMPGMMPPMTMMPMPSMMPSMVPTMMSPMTMASMMPPMMMPSMISPMTMPSMMPSMIMPTMMSPMIMPSMMPPMMMPSMVSPMMMPNMMTVPQCYSGSISHIIQQQQLPFMFSPTAMAIPPMFLQQPFVGAAF

SEQ ID NO: 2 - protein, 202 aa, synthetic construct
DAQTGTENLYFQSNAHIQALVTTTDAIGYHEPWMQYCMKQQGVANLLAWPTLMLQQLLASPLQQCQMPMMMPGMMPPMTMMPMPSMMPSMVPTMMSPMTMASMMPPMMMPSMISPMTMPSMMPSMIMPTMMSPMIMPSMMPPMMMPSMVSPMMMPNMMTVPQCYSGSISHIIQQQQLPFMFSPTAMAIPPMFLQQPFVGAAF

SEQ ID NO: 3 - protein, 163 aa, synthetic construct
QQGVANLLAWPTLMLQQLLASPLQQCQMPMMMPGMMPPMTMMPMPSMMPSMVPTMMSPMTMASMMPPMMMPSMISPMTMPSMMPSMIMPTMMSPMIMPSMMPPMMMPSMVSPMMMPNMMTVPQCYSGSISHIIQQQQLPFMFSPTAMAIPPMFLQQPFVGAAF

SEQ ID NO: 4 - protein, 233 aa, synthetic construct
MHHHHHHSTSVDLGTENLYFQSNAMAAKMFALFALLALCATATSATHIQALVTTTDAIGYHEPWMQYCMKQQGVANLLAWPTLMLQQLLASPLQQCQMPMMMPGMMPPMTMMPMPSMMPSMVPTMMSPMTMASMMPPMMMPSMISPMTMPSMMPSMIMPTMMSPMIMPSMMPPMMMPSMVSPMMMPNMMTVPQCYSGSISHIIQQQQLPFMFSPTAMAIPPMFLQQPFVGAAF

SEQ ID NO: 5 - DNA, 38 bp, synthetic construct
  source 1..38: mol_type = other DNA
tacttccaatccaatgcgcatatccaggcacttgtcac

SEQ ID NO: 6 - DNA, 38 bp, synthetic construct
  source 1..38: mol_type = other DNA
ttatccacttccaatgctagaatgcagcaccaacaaag

SEQ ID NO: 7 - DNA, 630 bp, synthetic construct
  source 1..630: mol_type = other DNA
atggcagccaagatgtttgcattgtttgcgctcctagctctttgtgcaaccgccactagtgctacccatatccaggcacttgtcaccactactgatgccattggctaccatgaaccatggatgcagtactgcatgaagcaacagggggttgccaacttgttagcgtggccgaccctgatgctgcagcaactgttggcctcaccgcttcagcagtgccagatgccaatgatgatgccgggtatgatgccaccgatgacgatgatgccgatgccgagtatgatgccatcgatggtgccgactatgatgtcaccaatgacgatggctagtatgatgccgccgatgatgatgccaagcatgatttcaccaatgacgatgccgagtatgatgccttcgatgataatgccgaccatgatgtcaccaatgattatgccgagtatgatgccaccaatgatgatgccgagcatggtgtcaccaatgatgatgccaaacatgatgacagtgccacaatgttactctggttctatctcacacattatacaacaacaacaattaccattcatgttcagccccacagccatggcgatcccacccatgttcttacagcagccctttgttggtgctgcattctag

SEQ ID NO: 8 - protein, 209 aa, synthetic construct
MAAKMFALFALLALCATATSATHIQALVTTTDAIGYHEPWMQYCMKQQGVANLLAWPTLMLQQLLASPLQQCQMPMMMPGMMPPMTMMPMPSMMPSMVPTMMSPMTMASMMPPMMMPSMISPMTMPSMMPSMIMPTMMSPMIMPSMMPPMMMPSMVSPMMMPNMMTVPQCYSGSISHIIQQQQLPFMFSPTAMAIPPMFLQQPFVGAAF

SEQ ID NO: 9 - DNA, 5247 bp, synthetic construct
  source 1..5247: mol_type = other DNA
cgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatttcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacacaaaccgcctctccccgcgcgttggccgattcattaatgcagctggcacgacaggtttcccgactggaaagcgggcagtgagcgcaacgcaattaatgtaagttagctcactcattaggcacaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagccagtccgtttaggtgttttcacgagcaattgaccaacaaggaccatagattatgaaaatccaccatcaccaccaccacgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagcagcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcggggagaacctgtacttccagatgctgatgggcggccgcgatatcgtcgacggatccgaattccctgcaggtaattaaataagcttcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttcggtccgttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgtttcccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttccttaggactgagcgtcaaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtgggtgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatataaggtgcactgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgcagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagctcaggccaggacccaacgctgcccgaaattc

SEQ ID NO: 10 - DNA, 1242 bp, synthetic construct
  source 1..1242: mol_type = other DNA
atgaaaatccaccatcaccaccaccacgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagcagcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcggggagaacctgtacttccagatgctgatgggcggccgcgatatcgtcgacggatccgaattccctgcaggtaattaa

SEQ ID NO: 11 - protein, 413 aa, synthetic construct
MKIHHHHHHEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGENLYFQMLMGGRDIVDGSEFPAGN

SEQ ID NO: 12 - protein, 612 aa, synthetic construct
MKIHHHHHHEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGENLYFQMLMGGRDIMAAKMFALFALLALCATATSATHIQALVTTTDAIGYHEPWMQYCMKQQGVANLLAWPTLMLQQLLASPLQQCQMPMMMPGMMPPMTMMPMPSMMPSMVPTMMSPMTMASMMPPMMMPSMISPMTMPSMMPSMIMPTMMSPMIMPSMMPPMMMPSMVSPMMMPNMMTVPQCYSGSISHIIQQQQLPFMFSPTAMAIPPMFLQQPFVGAAF

SEQ ID NO: 13 - protein, 205 aa, synthetic construct
MFALFALLALCATATSATHIQALVTTTDAIGYHEPWMQYCMKQQGVANLLAWPTLMLQQLLASPLQQCQMPMMMPGMMPPMTMMPMPSMMPSMVPTMMSPMTMASMMPPMMMPSMISPMTMPSMMPSMIMPTMMSPMIMPSMMPPMMMPSMVSPMMMPNMMTVPQCYSGSISHIIQQQQLPFMFSPTAMAIPPMFLQQPFVGAAF